(19) United States
(12) Patent Application Publication
(10) Pub. No.: US 2025/0059522 A1
(43) Pub. Date: Feb. 20, 2025

(54) SYNTHETIC REVERSE TRANSCRIPTASES AND USES THEREOF

(71) Applicant: SOLIS BIODYNE OÜ, Tartu (EE)

(72) Inventor: Olev KAHRE, Tartu (EE)

(21) Appl. No.: 18/630,879

(22) Filed: Apr. 9, 2024

Related U.S. Application Data
(63) Continuation of application No. 17/329,358, filed on May 25, 2021, now Pat. No. 11,987,822.
Application No. 17/329,358 is a continuation of application No. 16/092,753, filed on Oct. 10, 2018, now Pat. No. 11,046,940.
(60) Provisional application No. 62/321,692, filed on Apr. 12, 2016.

(51) Int. Cl. C12N 9/12; C12N 9/22

(52) U.S. Cl. CPC C12N 9/1276; C12N 9/22; C12Y 207/07049; C12Y 301/26004; C07K 2319/85

(57) ABSTRACT

The present disclosure provides non-natural reverse transcriptases for conducting reverse transcription. The non-natural reverse transcriptases herein may have increased thermostability and can conduct reverse transcription more efficiently than natural reverse transcriptases.

### CROSS-REFERENCE

This application is a continuation of U.S. patent application Ser. No. 17/329,358, filed May 25, 2021, which is a continuation of U.S. patent application Ser. No. 16/092,753, filed Oct. 10, 2018, now U.S. Pat. No. 11,046,940, issued Jun. 29, 2021, which is a U.S. National Stage entry under 35 U.S.C. § 371 of International Patent Application No.: PCT/IB2017/052114, filed Apr. 12, 2017, which claims the benefit of U.S. Provisional Application No. 62/321,692, filed Apr. 12, 2016, which applications are each incorporated herein by reference.

### SEQUENCE LISTING

The instant application contains a Sequence Listing which has been submitted electronically in XML format and is hereby incorporated by reference in its entirety. Said XML copy, created on Apr. 9, 2024, is named 39328-703_302SL.xml and is 43,054 bytes in size.

### BACKGROUND

Retroviruses replicate their genomes through reverse transcription, which is a step-wise process that leads to the synthesis of double-stranded DNA from a single stranded RNA molecule. Retroviral reverse transcription is generally accomplished by a viral reverse transcriptase that possesses DNA polymerase activity and ribonuclease (RNase) activity. The DNA polymerase has RNA-dependent DNA polymerase activity and may also possess DNA-dependent DNA polymerase activity, albeit with variable efficiency. The first step of reverse transcription is catalyzed by an RNA-dependent DNA polymerase that synthesizes single-stranded DNA using RNA as a template. The RNA portion of the resulting DNA-RNA hybrid is then degraded by the RNase H portion of the reverse transcriptase (RT). The resulting single-stranded DNA may be used as a template to produce double-stranded DNA, a process catalyzed by DNA-dependent DNA polymerase activity of the viral RT, if present, or by an exogenous DNA-dependent DNA polymerase, such as a DNA polymerase present in the host cell.

In a viral RT, the RNase domain (e.g., RNase H) is generally linked to the DNA polymerase domain. However, ribonucleases can also exist independently of reverse transcriptases. For example, cellular RNase Hs, which have been identified in various microorganisms such as bacteria (e.g., mesophilic and thermophilic) and archaea (e.g., thermophilic), can exist as independent molecules.

Natural viral RTs can be inefficient for various reasons. RTs, in some cases, can degrade an RNA template before the first strand reaction is initiated or completed, e.g., due to the intrinsic ribonuclease activity present in the enzyme. In addition, mis-priming of the RNA template molecule can lead to the introduction of errors in the cDNA first strand. Reverse transcription may be especially challenging when the template RNA has a high degree of secondary structure, which can occur, for example, when complementary regions within an RNA molecule hybridize to form double-stranded RNA. Generally, the detrimental effects of RNA secondary structure may be reduced by heating the RNA in a pre-incubation step prior to the initiation of reverse transcription.

There is a need in the art for a new RT with improved specific activity, efficiency, reaction speed, and/or stability, particularly at elevated temperatures. Novel approaches for generating new RTs are also needed.

### SUMMARY

This disclosure provides non-natural reverse transcriptases (non-natural “RTs”) with enhanced properties such as the ability to conduct reverse transcription at elevated temperatures with a high specific activity. This disclosure also provides methods of using the non-natural RTs, methods of generating the non-natural RTs and kits containing the non-natural RTs as one of the components of the kit.

In an aspect, the present disclosure provides a non-natural reverse transcriptase comprising a first domain and a second domain, wherein (a) the first domain comprises an enzyme with an amino acid sequence at least about 80% identical to an amino acid sequence of an enzyme from a first organism, and (b) the second domain comprises a modified ribonuclease polypeptide with an amino acid sequence between 50% and 99.9% identical to an amino acid sequence of a wild-type ribonuclease from a second organism; wherein the second organism is different from the first organism and wherein the modified ribonuclease polypeptide has reduced ribonuclease activity relative to ribonuclease activity of the wild-type ribonuclease from the second organism.

In an aspect, the present disclosure provides a non-natural reverse transcriptase comprising a first domain and a second domain, wherein (a) the first domain comprises an enzyme with an amino acid sequence at least about 80% identical to an amino acid sequence of an enzyme from a first organism, and (b) the second domain comprises a modified ribonuclease polypeptide with an amino acid sequence between 50% and 97% identical to an amino acid sequence of a wild-type ribonuclease from a second organism; wherein the second organism is different from the first organism and wherein the modified ribonuclease polypeptide has reduced ribonuclease activity relative to ribonuclease activity of the wild-type ribonuclease from the second organism.

In an aspect, the present disclosure provides a non-natural reverse transcriptase comprising a first domain and a second domain wherein (a) the first domain comprises an enzyme, and (b) the second domain comprises a modified ribonuclease polypeptide; wherein the first and second domains are not derived from the same organism and wherein the modified ribonuclease polypeptide comprises an amino acid sequence designed to reduce activity of the modified ribonuclease polypeptide when compared to a naturally-occurring version of the modified ribonuclease polypeptide.

In some embodiments, the modified ribonuclease polypeptide comprises an RNase H polypeptide. In some embodiments, the RNase H polypeptide comprises a mutated RNase H domain. In some embodiments, the mutated RNase H domain comprises at least one mutation in an active site. In some embodiments, the at least one mutation in the active site decreases RNase H activity of the RNase H domain relative to an un-mutated version of the RNase H domain. In some embodiments, the at least one mutation is an amino acid substitution, insertion, or deletion. In some embodiments, the non-natural reverse transcriptase has at most 75%, 50%, 25%, 10%, 1%, 0.1%, or less of the ribonuclease activity of a wild type version of the modified ribonuclease polypeptide.

In some embodiments, the first domain is linked to the second domain. In some embodiments, the first domain is linked to the N-terminus of the modified ribonuclease polypeptide. In some embodiments, a non-natural reverse transcriptase provided herein further comprises a third polypeptide. In some embodiments, the third polypeptide comprises a peptide tag that confers thermal stability to the non-natural reverse transcriptase. In some embodiments, the third polypeptide is positioned at the N-terminus of the non-natural reverse transcriptase. In some embodiments, the third polypeptide is positioned at the C-terminus of the non-natural reverse transcriptase. In some embodiments, the non-natural reverse transcriptase further comprises a fourth polypeptide. In some embodiments, the fourth polypeptide comprises a peptide tag that confers thermal stability to the non-natural reverse transcriptase. The fourth polypeptide can be positioned at the N-terminus or at the C-terminus.

In some embodiments, the enzyme comprises a polymerase domain or a variant thereof. In some embodiments, the enzyme comprises a polymerase and the polymerase is derived from a virus, an avian virus, a human virus, a murine virus, a retrovirus, a lentivirus, or a gammaretrovirus. In some embodiments, the polymerase domain or variant thereof is derived from a viral reverse transcriptase selected from the group consisting of: Moloney Murine Leukemia Virus (M-MLV) reverse transcriptase, Rous Sarcoma Virus (RSV) reverse transcriptase, Avian Myeloblastosis Virus (AMV) reverse transcriptase, Myeloblastosis Associated Virus (MAV) reverse transcriptase, Rous Associated Virus (RAV) reverse transcriptase, and Human Immunodeficiency Virus 1 (HIV-1) reverse transcriptase. In some embodiments, the enzyme comprises a polymerase domain or a variant thereof, and the polymerase domain is derived from a virus, an avian virus, a human virus, a murine virus, a retrovirus, a lentivirus, or a gammaretrovirus. In some embodiments, the polymerase or polymerase domain or variant thereof comprises an amino acid sequence of (or is derived from) a polymerase domain of a viral reverse transcriptase selected from the group consisting of Moloney Murine Leukemia Virus (M-MLV) reverse transcriptase, Rous Sarcoma Virus (RSV) reverse transcriptase, Avian Myeloblastosis Virus (AMV) reverse transcriptase, Myeloblastosis Associated Virus (MAV) reverse transcriptase, Rous Associated Virus (RAV) reverse transcriptase, and Human Immunodeficiency

In some embodiments, the enzyme is derived from a polymerase and the polymerase is a DNA polymerase. In some embodiments, the enzyme is derived from a polymerase and the polymerase is an RNA-dependent DNA polymerase. In some embodiments, the enzyme is derived from a polymerase and the polymerase is a DNA-dependent DNA polymerase. In some embodiments, the polymerase is derived from an M-MLV reverse transcriptase. In some embodiments, the polymerase is derived from HIV-1 reverse transcriptase. In some embodiments, the enzyme is a polymerase selected from the group consisting of DNA polymerase, RNA-dependent DNA polymerase, and DNA-dependent DNA polymerase. In some embodiments, the polymerase is derived from a viral reverse transcriptase. In some embodiments, the viral reverse transcriptase is selected from the group consisting of M-MLV reverse transcriptase and HIV-1 reverse transcriptase.

In some embodiments, the modified ribonuclease polypeptide is derived from a bacterial RNase H or an archaeal RNase H. In some embodiments, the modified ribonuclease polypeptide is derived from an extremophile organism. In some embodiments, the modified ribonuclease polypeptide is derived from a polypeptide selected from the group consisting of: Pyrococcus furiosus RNase H, Pyrococcus horikoshi RNase H, Thermococcus litoralis RNase H II, Thermus thermophilus RNase H, RNase H, RNase HI, Rnase HII, RNase HIII, and Escherichia coli RNase H. In some embodiments, the modified ribonuclease polypeptide is derived from an organism of the genus Thermus. In some embodiments, the modified ribonuclease polypeptide is derived from Thermus thermophilus RNase H. In some embodiments, the modified ribonuclease polypeptide comprises an amino acid sequence at least 85%, 90%, or 95% identical to SEQ ID NO: 21. In some embodiments, the modified ribonuclease polypeptide is derived from an organism of the genus Thermococcus. In some embodiments, the modified ribonuclease polypeptide is derived from Thermococcus litoralis RNase H. In some embodiments, the modified ribonuclease polypeptide comprises an amino acid sequence at least 85%, 90%, or 95% identical to SEQ ID NO: 22. In some embodiments, the modified ribonuclease polypeptide comprises an active site comprising a mutation at at least one of residues 14 (D), 52 (E), 74 (D) and 139 (D) of SEQ ID NO: 7. In some embodiments, the modified ribonuclease polypeptide comprises an active site comprising a mutation at at least one of residues 7 (D), 8 (E), 105 (D) and 135 (D) of SEQ ID NO: 8.

In some embodiments, the non-natural reverse transcriptase retains greater than 50% of its reverse transcriptase activity at temperatures above 55° C. In some embodiments, the non-natural reverse transcriptase retains greater than 50% of its reverse transcriptase activity after incubation at a temperature of least about 55° C. or higher for at least 15 minutes. In some embodiments, the non-natural reverse transcriptase retains reverse transcriptase activity after incubation at a temperature of least about 60° C. or higher for at least 15 minutes. In some embodiments, the non-natural reverse transcriptase retains reverse transcriptase activity after incubation at a temperature of least about 65° C. or higher for at least 15 minutes. In some embodiments, the non-natural reverse transcriptase retains reverse transcriptase activity after incubation at a temperature between 40° C. and 65° C., at a temperature between 60° C. and 65° C., at a temperature between 55° C. and 60° C. or at a temperature between 55° C. and 75° C. In some cases, such incubation is for 15 minutes, 30 minutes, 1 hour, or longer. In some cases, such incubation lasts between 15 minutes and 2 hours, between 15 minutes and 1 hour, between 30 minutes and 2 hours, or between 60 minutes and 2 hours.

In some embodiments, the non-natural reverse transcriptase comprises an amino acid sequence at least 85% identical to SEQ ID NO: 9, SEQ ID NO: 10, SEQ ID NO: 11, SEQ ID NO: 12, SEQ ID NO: 14, SEQ ID NO: 15, SEQ ID NO: 16 or SEQ ID NO: 17. In some embodiments, the non-natural reverse transcriptase has a specific activity of at least about 30,000 U/μg.

In an aspect, the present disclosure provides a non-natural reverse transcriptase comprising (a) a first domain comprising an enzyme, and (b) a second domain comprising a ribonuclease polypeptide having ribonuclease activity; wherein the non-natural reverse transcriptase retains at least 25% of its reverse transcriptase activity at about 37° C. after incubation at a temperature of at least about 60° C. for at least 10 minutes.

In some embodiments, the first domain is linked to the second domain.

In some embodiments, the specific activity of the non-natural reverse transcriptase is greater than 450 U/μg. In some embodiments, the specific activity of the non-natural reverse transcriptase is greater than 10,000 U/μg. In some embodiments, the specific activity of the non-natural reverse transcriptase is greater than 20,000 U/μg. In some embodiments, the specific activity of the non-natural reverse transcriptase is greater than 30,000 U/μg.

In some embodiments, the second domain is derived from an extremophile organism.

In an aspect, the present disclosure provides a kit for complementary DNA (cDNA) synthesis comprising any non-natural reverse transcriptase provided herein. In some cases, the kit further comprises a DNA-dependent DNA polymerase in a separate container from the non-natural reverse transcriptase. In some cases, the kit further comprises a DNA-dependent DNA polymerase mixed in the same container as the non-natural reverse transcriptase.

In some embodiments, the kit further comprises a primer to initiate cDNA synthesis. In some embodiments, the primer is an oligo(dT) primer. In some embodiments, the kit further comprises dNTPs. In some embodiments, the kit further comprises a reaction buffer. In some embodiments, the reaction buffer comprises divalent metal ions. In some embodiments, the divalent metal ions are Mg2+ or Mn2+.

In some embodiments, the kit is stored at room temperature.

In an aspect, the present disclosure provides a method for synthesizing complementary DNA (cDNA), comprising (a) providing an RNA molecule as a template for cDNA synthesis, (b) providing a primer to initiate cDNA synthesis from the RNA molecule, and (c) synthesizing cDNA initiated by the primer from the template using any non-natural reverse transcriptase provided herein.

In an aspect, the present disclosure provides a method of synthesizing a reverse transcriptase comprising linking an enzyme to a ribonuclease polypeptide, wherein the enzyme and the ribonuclease are from different organisms, and wherein the ribonuclease is specifically selected to increase the thermal stability of the reverse transcriptase.

In some embodiments, the ribonuclease is derived from an extremophile RNase. In some embodiments, the ribonuclease is derived from a bacterial RNase or an archaeal RNase H. In some embodiments, the ribonuclease is derived from an RNase selected from the group consisting of: Pyrococcus furiosus RNase H, Pyrococcus horikoshi RNase H, Thermococcus litoralis RNase H II, Thermus thermophilus RNase H, and Escherichia coli RNase H. In some embodiments, the RNase H is Thermus thermophilus RNase H. In some embodiments, the RNase H is Thermococcus litoralis RNase H.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows DNA product from reverse transcription performed with non-natural reverse transcriptases provided herein and subsequent amplification via a DNA polymerase.

FIG. 2 shows the molecular weight of non-natural reverse transcriptases on a protein gel.

FIG. 3 shows DNA product from reverse transcription performed with non-natural reverse transcriptases following pre-incubation at elevated temperatures and subsequent amplification via a DNA polymerase.

FIGS. 4A and 4B show the amplification product from first strand cDNA synthesis and PCR amplification using wild-type M-MLV RT and a chimeric RT disclosed herein with different amounts of template.

### DETAILED DESCRIPTION

### Overview

The present disclosure provides compositions comprising non-natural chimeric RTs, methods for conducting reverse transcription using the non-natural RTs, methods for designing and generating the non-natural RTs and kits comprising the non-natural RTs. Generally, the non-natural RTs provided herein comprise at least two domains selected to impart certain benefits on the molecule (or complex of molecules, referred to interchangeably as “molecule complex”) as a whole. The resulting molecule (or molecule complex) may have features such as particularly high thermal stability, specific activity, sensitivity, efficiency, reaction speed, or any combination thereof. Such features may also be particularly robust in comparison to naturally-occurring RTs.

In some examples, a RT provided herein comprises (a) a reverse transcriptase domain specifically selected from a species of virus known to have some amount of RNA-dependent DNA polymerase activity (e.g., weak, moderate, or high) and (b) a RNase domain (e.g., RNase H domain) selected from a species of bacteria or archaea known to have high temperature tolerance or other attributes (e.g., extremophile bacterial RNase H, extremophile archaean RNase H). In some examples, a RT provided herein comprises (a) a reverse transcriptase domain specifically selected from a species of virus known to have efficient RNA-dependent DNA polymerase activity (e.g., M-MLV) and (b) a RNase domain (e.g., RNase H domain) selected from a species of bacteria or archaea known to have high temperature tolerance or other attributes (e.g., extremophile bacterial RNase H, extremophile archaean RNase H). The RNA-dependent DNA polymerase activity may be determined by any suitable assay or comparison to any accepted standard or metric, for example, a specific activity measurement. In some cases, the endogenous RNase domain of the RT may possess RNase activity (e.g., RNase H activity); while, in others, the RT possesses minimal or no RNase activity. A decrease or lack of RNase activity in an RT can result, for example, from inactivating mutations introduced into the RNase domain. An RT provided herein may also possess DNA-dependent DNA polymerase activity; while, in other cases, the RT possesses minimal or no DNA-dependent polymerase activity. In some cases, the non-natural RT possesses both RNA-dependent and DNA-dependent polymerase activity. In some cases, the RT possesses RNA-dependent DNA polymerase activity but not DNA-dependent polymerase activity.

The RTs provided herein may have robust thermal stability, which may provide a number of advantages. Thermal stability can refer to the stability of the structure or activity of a protein as a function of temperature. Generally, increases in temperature can result in changes to the structure of a protein which consequently may change properties of the protein related to its structure, for example enzymatic activity in the case of enzymes and/or the ability to interact with binding partners. RTs with increased thermal stability, for example, may enable reduction of the number of steps required to complete reverse transcription, thereby reducing the total reaction time, or the time from single-stranded RNA to double-stranded cDNA. RNA templates, particularly RNA templates with high degrees of secondary structure, may require a pre-incubation step at an elevated temperature in order to denature the RNA, followed by a cooling step in order to cool the sample to a temperature that would not interfere with the function of a non-thermally stable RT. In contrast, a thermal-stable RT provided herein can be included with a sample during the pre-incubation step; and the cooling step may be skipped since the RT can still function at the higher temperature. Another advantage of the RTs provided herein is that they may be used to conduct reverse transcription at elevated temperatures.

The non-natural RTs provided herein may have particularly high RNA-directed DNA polymerase specific activity. For example, a non-natural RT provided herein may have a RNA-directed DNA polymerase specific activity of at least 450 U/μg. In some cases, a non-natural RT provided herein may have a RNA-directed DNA polymerase specific activity of at least 100 U/μg, at least 500 U/μg, at least 1,000 U/μg, at least 5,000 U/μg, at least 10,000 U/μg, at least 15,000 U/μg, at least 20,000 U/μg, at least 25,000 U/μg, at least 30,000 U/μg, or greater. In some cases, the specific activity may be particularly high in comparison to a naturally-occurring RT. For example, a non-natural RT provided herein may have greater than 3-fold specific activity compared to its wild-type form. In some cases, a non-natural RT provided herein may have at least 5-fold, 10-fold, 20-fold, 30-fold, 40-fold, 50-fold, at least 100-fold, at least 150-fold, or greater specific activity compared to its wild-type form.

The non-natural RTs provided herein may also display highly efficient reverse transcription or the conversion of RNA into cDNA. The non-natural RTs may more efficiently convert mRNA into cDNA in reverse transcription due to enhanced thermal stability, decreased ribonuclease activity and/or increased polymerase activity compared to wild-type RTs. In some cases, a non-natural or chimeric reverse transcriptase provided herein may have an efficiency that is at least about 0.1-fold, 0.2-fold, 0.3-fold, 0.4-fold, 0.5-fold, 1-fold, 2-fold, 3-fold, 4-fold, 5-fold, 10-fold or greater than 10-fold the efficiency of its wild-type or natural form. In some cases, a non-natural or chimeric reverse transcriptase provided herein may have an efficiency that is at least about 20-fold, 30-fold, 40-fold, 50-fold, 60-fold, 70-fold, 80-fold, 90-fold, 100-fold, 110-fold, 120-fold, 130-fold, 140-fold, 150-fold, 160-fold, 170-fold, 180-fold, 190-fold, 200-fold, 250-fold, 300-fold, 350-fold, 400-fold, 450-fold, 500-fold, or greater than 500-fold the efficiency of its wild-type or natural form. In some cases, a non-natural or chimeric reverse transcriptase provided herein may have an efficiency that is between 5-fold and 400-fold the efficiency of its wild-type of natural form.

The compositions provided herein include compositions comprising nucleic acids encoding a non-natural chimeric RT provided herein. In some cases, the nucleic acids encode an entire RT protein. In some cases, the nucleic acids encode a domain (e.g., polymerase domain, RNase domain) of an RT protein.

### Domains

Generally, the non-natural chimeric RTs provided herein comprise at least two domains, such as at least one DNA polymerase domain (e.g., DNA-dependent DNA polymerase or RNA-dependent DNA polymerase) and at least one RNase domain. The term “domain,” as used herein, generally refers to a discrete set or sequence of units (e.g., nucleic acids, amino acids, etc) within a polymer such as a polynucleotide, polypeptide or other macromolecule. The discrete set of units can be a consecutive sequence of units (e.g., units at positions 4-10 of a macromolecule). In some cases, the discrete set may also comprise units (e.g., amino acids) that are disparately or sporadically positioned within the macromolecule (e.g, positioned at positions 4, 7, 8, 9, 11, and 12 of the macromolecule). A domain may also refer to a region of a macromolecule that is close together in three-dimensional space (e.g., a binding domain), often due to folding (e.g., protein folding); such region may or may not be associated with a specific sequence of units, e.g., amino acid sequence. A domain often may be associated with a particular activity or function, such as DNA polymerase activity or RNase activity. In some cases, a domain is associated with more than one activity or function, e.g., both RNA-dependent DNA polymerase activity and DNA-dependent DNA polymerase activity. In some cases, a particular domain may refer to a discrete set or sequence of units (e.g., amino acids) with no particular function.

A polypeptide domain provided herein may be encoded by a single polynucleotide. A polypeptide domain can be encoded by a portion of a polynucleotide that encodes other domains within the polypeptide.

The RTs provided herein may have any number of domains, e.g., at least about 1 domain, at least about 2 domains, at least about 3 domains, at least about 4 domains, at least about 5 domains, at least about 6 domains, at least about 7 domains, at least about 8 domains, at least about 9 domains, at least about 10 domains, at least about 20 domains, or more than 20 domains. In some cases, two or more domains within the RT are identical. In some cases, two or more domains within the RT have >50% sequence identity. In some cases, two or more domains in the RT have a different function, sequence, and/or structure.

Two or more domains within a RT provided herein may be present within a single polypeptide (e.g., as a monomeric polypeptide). In some cases, two or more domains of an RT provided herein are present within multiple polypeptides, or multiple subunits. For example, at least one domain within a RT provided herein is present in one subunit of the RT and at least one domain within the RT is located in a different subunit. The subunits can associate together to form a single complex. However, in some cases, the subunits are located in different complexes. For example, a DNA polymerase domain (e.g., RNA-dependent DNA polymerase) may be present in one unit of a complex and the RNase domain may be present in a different unit within a complex, or in a separate molecule or complex altogether.

### Chimeric Reverse Transcriptases

The non-natural RTs provided herein are generally chimeric proteins and thus may be referred to as “chimeric RTs”. As used herein, the terms “chimeric protein” or “chimeric polypeptide” may be used interchangeably to refer to a protein comprising two or more polypeptide sequences or domains that are not naturally present in the same polypeptide. In some cases, the two or more polypeptide sequences or domains may be from the same type of organism. In some cases, the two or more polypeptide sequences or domains may be from at least two different types of organisms. In some cases, the two or more polypeptide sequences or domains may be derived from two polypeptide domains that occur in nature, except that one or more of the peptides is altered so as not to markedly resemble a naturally-occurring domain (e.g., by mutation). For example, the altered peptide can be less than 100%, less than 90%, less than 80%, less than 70%, less than 60%, less than 50%, less than 40%, less than 30%, less than 20%, less than 10% or less than 5% identical to the naturally-occurring domain. In some examples, the two or more polypeptide sequences or domains are derived from a virus. In some instances, the two or more polypeptide sequences or domains derived from a virus are derived from the same family of virus (e.g., retrovirus), or the same genus of virus (e.g., gammaretrovirus), or the same type of virus (e.g., Murine Leukemia Virus (MLV)); or the same strain or subtype of virus (e.g., Moloney Murine Leukemia Virus (M-MLV or M-MuLV)). In some cases, the two or more polypeptide sequences or domains are derived from the same domain (e.g., biological classification, phylogeny) (e.g., Archaea). In some cases, the two or more polypeptide sequences or domains are derived from the same kingdom (e.g., Euryarchaeota). In some cases, the two or more polypeptide sequences or domains are derived from the same family (e.g., Thermaceae, Thermococcaceae); from the same genus (e.g., Thermus, Thermococcus); or from the same species (e.g., Thermus thermophilus, Thermococcus litoralis). In some cases, the two or more polypeptide sequences or domains are derived from the same class (e.g., Deinococci, Thermocci); or from the same order (e.g., Thermales, Thermococcales).

In some embodiments, the two or more polypeptide sequences or domains within the RTs provided herein are derived from different organisms (e.g., one may be from a virus, the other from a bacterium or archaeon). In some cases, the two or more polypeptide sequences or domains are derived from different kingdoms; from different phylums; from different classes; from different orders; from different families; from different genuses; from different species; from different strains; or from different subtypes.

In some cases, the RTs provided herein comprise at least one domain or sequence derived from a retrovirus (e.g., MLV, M-MLV, AMV) and at least one domain or sequence derived from a bacterium or archaeon (e.g., extremophile bacterium, extremophile archaeon, Thermus bacterium, Thermococcus archaea, T. thermophilus, T. thermophilus HB8, T. litoralis, etc), in any combination thereof. In various embodiments, the DNA polymerase domain (or domains) is derived from a virus and the RNase domain (or domains) is derived from a bacterium or archaeon. The RT may comprise an M-MLV domain (e.g., an M-MLV DNA polymerase domain) and an extremophile domain (e.g., RNase domain from an extremophilic bacterium or from an extremophilic archaeon). In some cases, the RT may comprise a MLV domain (e.g., an MLV DNA polymerase domain, an M-MLV DNA polymerase domain) and a Thermus bacterium domain (e.g., RNase from T. Thermophilus). In some embodiments, the RTs provided herein comprise at least one domain or sequence derived from a virus (e.g., retrovirus, e.g., MLV, M-MLV, AMV) and at least one domain or sequence derived from an archaeon (e.g., extremophile archaeon, T. litoralis) in any combination. Generally, the DNA polymerase domain (or domains) is derived from a virus. Generally, the RNase domain (or domains) is derived from a bacterium or archaeon organism. For example, the RT may comprise a MLV domain (e.g., an MLV DNA polymerase domain, particularly an M-HLV DNA polymerase domain) and an RNase domain derived from a Thermus bacterium (e.g., RNaseH domain from T. Thermophilus). For further example, the RTs provided herein may comprise a MLV domain (e.g., an MLV DNA polymerase domain) and an RNase domain derived from a Thermococcus archaeon (e.g., RNaseH domain from T. litoralis).

An RNase domain within an RT provided herein may be selected because it is known or suspected to share certain structural and/or functional features with an RNase from a different organism (or strain, species, genus, family, order, class, domain, etc.). In some cases, the RNase domain is derived from an organism (or strain, species, genus, family, order, class, domain, etc.) different from the DNA polymerase domain within the RT but is known or suspected to share certain structural and/or functional features with the RNase of the wild-type or natural RT. In some instances, a T. thermophilus RNase H may be homologous to an RNase (e.g., RNase H) domain from M-MLV in terms of sequence and/or structure (e.g., 3D structure). For example, the RNase (e.g., RNase H) domain of an RT provided herein may be a T. thermophilus RNase and the DNA polymerase domain of the RT may be from M-MLV. In another example, the T. thermophilus RNase may be combined with an HIV DNA polymerase, or other DNA polymerase provided herein. In still another example, an E. Coli RNase may be combined with an M-MLV or HIV DNA polymerase.

In some cases, an RNase domain within an RT provided herein may be selected because it is known or suspected to possess dissimilar structural and/or functional features compared to an RNase from a different organism (or strain, species, genus, family, order, class, domain, etc.). In some cases, the RNase domain is derived from an organism (or strain, species, genus, family, order, class, domain, etc.) different from the DNA polymerase domain within the RT but is known or suspected to possess dissimilar structural and/or functional features with the RNase of the wild-type or natural RT. For example, T. litoralis RNase H II may lack homology to an RNase H from M-MLV or HIV in terms of sequence and/or structure (e.g., 3D structure). For example, the RNase H domain of an RT provided herein may be a T. litoralis RNase H II and the DNA polymerase domain of the RT may be from M-MLV or HIV.

An RT provided herein may comprise a retroviral DNA polymerase domain and an RNase domain derived from a cellular microorganism that exhibits a certain feature, such as heat tolerance or thermal stability. The microorganism may be a thermophilic microorganism such as a thermophilic bacterium or archaeon (e.g., T. thermophilus or T. litoralis). The resulting chimeric RT may have enhanced thermostability when compared to a naturally-occurring RT, or to a RT in which all of the domains are derived from a retrovirus. Additional thermostable microorganisms include, but are not limited to, Thermococcus gammatolerans, Thermus flavus, Thermus ruber, Thermus thermophilus, Bacillus stearothermophilus, Thermus lacteus, Thermus rubens, Thermotoga maritima, and Methanothermus fervidus. In some cases, the thermostable microorganism is radiation-resistant.

In some cases, a chimeric reverse transcriptase may comprise at least two domains that are derived from viruses. For example, the RT may comprise the polymerase domain of M-MLV RT linked to the ribonuclease domain of HIV-1 RT. Conversely, a chimeric RT may comprise the polymerase domain of HIV-1 RT linked to the ribonuclease domain of M-MLV RT or AMV RT. In another example, a chimeric reverse transcriptase may comprise the polymerase domain of AMV RT linked to the ribonuclease domain of M-MLV RT or HIV-1 RT.

In some cases, the polymerase domain and/or the ribonuclease domain can be truncated at either an N-terminus or a C-terminus. Certain amino acids at the N-terminus or C-terminus of a domain may not be necessary for function and/or structure, and removal of these amino acids may help with, for example, formation (e.g., folding) of the chimeric protein while minimally affecting the function of the chimeric protein. In some cases, the polymerase domain and/or the ribonuclease domain can be truncated at the N-terminus relative to the wild-type sequence of the domain. The polymerase domain and/or ribonuclease domain derived from a viral reverse transcriptase can be truncated by at least 1 amino acid (e.g., at least 2, 3, 4, 5, 6, 7, 8 amino acids or more than 8 amino acids) at the N-terminus relative to the wild-type sequence of the domain. The ribonuclease domain derived from a bacterial or archaeal RNase can be truncated by at least 1 amino acid (e.g., at least 2, 3, 4, 5, 6, 7, 8 amino acids or more than 8 amino acids) at the N-terminus relative to the wild-type sequence of the domain. In some cases, the polymerase domain and/or the ribonuclease domain can be truncated at the C-terminus relative to the wild-type sequence of the domain. In some cases, the polymerase domain and/or ribonuclease domain derived from a viral reverse transcriptase can be truncated by at least 1 amino acid (e.g., at least 2, 3, 4, 5, 6, 7, 8 amino acids or more than 8 amino acids) at the C-terminus relative to the wild-type sequence of the domain. In some cases, the ribonuclease domain derived from a bacterial or archaeal RNase can be truncated by at least 1 amino acid (e.g., at least 2, 3, 4, 5, 6, 7, 8 amino acids or more than 8 amino acids) at the C-terminus relative to the wild-type sequence of the domain. Truncated polymerase and/or ribonuclease domains derived from viral reverse transcriptases may help with the folding of a chimeric RT disclosed herein while minimally affecting the activity and/or function of the protein.

In some embodiments, a non-natural reverse transcriptase may comprise mutations in one or more domains in order to enhance or reduce certain enzymatic activities. In some cases, at least 1 mutation (e.g., at least 2, 3, 4, 5, 6, 7, 8, 9, 10 or greater than 10 mutations) is present in one or more domains of a non-natural (e.g., chimeric) RT, for example in a polymerase domain or ribonuclease domain. Mutations in the one or more domains may be any mutation or variation known in the art including amino acid insertions, deletions, or substitutions, in the respective domains. A desired property of the RT, for example, may be increased efficiency of cDNA generation. Increased efficiency of cDNA generation can be accomplished, in some cases, by a reduction of ribonuclease activity. Increased efficiency of cDNA generation can be accomplished, in some cases, by an increase in DNA polymerase activity. A chimeric RT of the disclosure may have reduced or substantially reduced ribonuclease activity (e.g., RNase H activity) or in some cases, may completely lack ribonuclease activity. The reduction in ribonuclease activity can result from one or more mutations in an active site of the domain. A chimeric RT of the disclosure may have increased DNA polymerase activity. The increase in polymerase activity can result from one or more mutations in an active site of the domain. Active sites generally refer to a region of an enzyme where a substrate molecule binds and undergoes a chemical reaction. Active sites generally contain residues that form non-covalent and/or covalent interactions with substrates which are important for enzymatic activity. Changes at the interface of the active site can, in some cases, reduce or diminish enzymatic activities. Alternatively, mutations made in active sites may enhance or increase enzymatic activities depending on the type of mutation (e.g., conservative or non-conservative mutations). In some cases, at least one mutation is present in an active site. In some cases, mutations made to the domain other than at the active site can also result in changes in enzymatic activity. Such mutations may result in indirect changes to an active site, for example, changes to the conformation of the active such that it has decreased or increased ability to bind to a substrate.

A viral polymerase domain and/or a viral ribonuclease domain of a chimeric construct described herein may have one or more mutations compared to the natural or wild-type sequence of the domain. In some cases, the polymerase domain comprises at least 1 mutation (e.g., at least 2, 3, 4, 5, 6, 7, 8 mutations or more than 8 mutations) relative to the wild-type sequence of the domain. In some cases, the ribonuclease domain comprises at least 1 mutation (e.g., at least 2, 3, 4, 5, 6, 7, 8 mutations or more than 8 mutations) relative to the native sequence. For example, an M-MLV RT may comprise a D524G, E562Q, and/or D583N mutation as described in U.S. Pat. No. 8,753,845, which is herein incorporated in its entirety for all purposes. Such mutations may reduce RNase H activity.

The RTs provided herein may possess at least one non-mutated domain and at least one mutated domain in any combination. In some cases, the mutated domain is an RNase domain having a mutation that reduces or eliminates ribonuclease activity. For example, a non-natural RT provided herein (SEQ ID NO: 9) may comprise a mutated variant of a ribonuclease domain from T. thermophilus (e.g., SEQ ID NO: 21) linked to a polymerase domain from M-MLV RT (e.g., SEQ ID NO: 2). In some embodiments, the ribonuclease domain is not mutated (e.g., wild-type sequence). In some embodiments, a non-natural (e.g., chimeric) RT provided herein (SEQ ID NO: 10) comprises a mutated variant of a ribonuclease domain from T. litoralis (e.g., RNase H II) (e.g., SEQ ID NO: 22) linked to a polymerase domain from M-MLV RT (e.g., SEQ ID NO: 2). In some embodiments, the ribonuclease domain is not mutated (e.g., wild-type sequence). In some embodiments, a non-natural (e.g., chimeric) RT provided herein comprises a mutated variant of a ribonuclease domain from Thermococcus gammatolerans (T. gammatolerans) linked to a polymerase domain from M-MLV RT (SEQ ID NO: 10). In some embodiments, the ribonuclease domain is not mutated (e.g., wild-type sequence). In some embodiments, a non-natural (e.g., chimeric) reverse transcriptase (SEQ ID NO: 11) comprises a mutated variant of a ribonuclease domain from T. thermophilus (e.g., SEQ ID NO: 21) linked to a polymerase domain from HIV-1 RT (e.g., SEQ ID NO: 5). In some embodiments, the ribonuclease domain is not mutated (e.g., wild-type sequence). In some embodiments, a non-natural (e.g., chimeric) reverse transcriptase (SEQ ID NO: 12) comprises a mutated variant of a ribonuclease domain from T. litoralis (e.g., RNase H II) (e.g., SEQ ID NO: 22) linked to a polymerase domain from HIV-1 RT (e.g., SEQ ID NO: 5). In some embodiments, the ribonuclease domain is not mutated (e.g., wild-type sequence). In some embodiments, a non-natural (e.g., chimeric) RT provided herein comprises a mutated variant of a ribonuclease domain from Thermococcus gammatolerans (T. gammatolerans) linked to a polymerase domain from HIV-1 RT (SEQ ID NO: 11). In some embodiments, the ribonuclease domain is not mutated (e.g., wild-type sequence). In some embodiments, the mutations described herein result in a mutated variant having reduced ribonuclease activity. In some embodiments, the mutations described herein result in a mutated variant having no discernable ribonuclease activity.

In some cases, a chimeric reverse transcriptase may comprise an amino acid sequence of any of SEQ ID NOs: 9-12. In some cases, a chimeric reverse transcriptase may comprise an amino acid sequence at least 75%, at least 80%, at least 85%, at least 90%, at least 91%, at least 92%, at least 93%, at least 94%, at least 95%, at least 96%, at least 97%, at least 98%, or at least 99% identical to of any of SEQ ID NOs: 9-12.

### Polymerase Domain

A polymerase domain of an RT provided herein may comprise any polymerase or polymerase activity, e.g., DNA polymerase, RNA polymerase, DNA-dependent DNA polymerase, or RNA-dependent DNA polymerase. In some embodiments, the polymerase domain is a DNA polymerase. In some cases, the polymerase domain has one or more activities, e.g., DNA-dependent DNA polymerase activity and/or RNA-dependent DNA polymerase activity. In some cases, the polymerase domain has RNA-dependent DNA polymerase activity but not DNA-dependent DNA polymerase activity or limited DNA-dependent DNA polymerase activity.

In some cases, a polymerase domain within a non-natural RT provided herein may be at least about 80% identical (e.g., at least about 85%, 90%, 95% identical or 100% identical) to the polymerase domain of a viral reverse transcriptase, such as a Moloney Murine Leukemia Virus Reverse Transcriptase (M-MLV RT), Human Immunodeficiency Virus-1 Reverse Transcriptase (HIV-1 RT), or Avian Myeloblastosis Virus Reverse Transcriptase (AMV RT).

The activity of a polymerase domain within an RT provided herein may be increased, for example, by mutation (or genetic modification). A polymerase domain with increased polymerase activity may have at least about 2.5%, at least about 5%, at least about 7.5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% greater activity than the corresponding unmutated polymerase. In some cases, the polymerase domain may have decreased polymerase activity. Decreased polymerase activity can be acceptable, for example, if there are other improvements in the domain (e.g., improved folding, etc). In some embodiments, a chimeric RT of the disclosure may not contain a modification or mutation in the polymerase domain and may not contain a modification which increases or decreases polymerase activity.

A polymerase domain in a non-natural RT provided herein may comprise one or more truncations. For example, it may be truncated at the N-terminus, e.g., by at least 1 amino acid (e.g., at least 2, 3, 4, 5, 6, 7, 8 amino acids or more than 8 amino acids). In some cases, the polymerase domain within a non-natural RT provided herein may be truncated at the C-terminus (e.g., at least 2, 3, 4, 5, 6, 7, 8 amino acids or more than 8 amino acids).

A polymerase domain within in a non-natural RT provided herein may comprise one or more mutations compared to the wild-type sequence of the domain. In some cases, the polymerase domain comprises at least 1 mutation (e.g., at least 2, 3, 4, 5, 6, 7, 8 mutations or more than 8 mutations). In some cases, the ribonuclease domain may be at least 60% identical (e.g., at least 65%, 70%, 75%, 80%, 85%, 90%, or 95% identical) and less than 100% identical to a wild-type RNase domain.

In some cases, the polymerase domain is derived from one or more of the following proteins: retroviral reverse transcriptase, lentiviral reverse transcriptase, retrotransposon reverse transcriptase, hepatitis B reverse transcriptase, cauliflower mosaic virus reverse transcriptase, bacterial reverse transcriptase, Tth DNA polymerase, Taq DNA polymerase, Tne DNA polymerase, Tma DNA polymerase, and any mutant, fragment, variant or derivative thereof. Reverse transcriptases having polymerase domains include retroviral RTs such as Moloney Murine Leukemia Virus (M-MLV or M-MuLV) RT, Human Immunodeficiency Virus (e.g., HIV-1 and HIV-2) RT, Simian Immunodeficiency Virus (SIV) RT, Avian Sarcoma-Leukosis Virus (ASLV) RT, and Avian Myeloblastosis Virus (AMV) RT. Examples of ASLV RTs include, but are not limited to, Rous Sarcoma Virus (RSV) RT, Avian Myeloblastosis Virus (AMV) RT, Avian Erythroblastosis Virus (AEV) Helper Virus MCAV RT, Avian Myelocytomatosis Virus MC29 Helper Virus MCAV RT, Avian Reticuloendotheliosis Virus (REV-T) Helper Virus REV-A RT, Avian Sarcoma Virus UR2 Helper Virus UR2AV RT, Avian Sarcoma Virus Y73 Helper Virus YAV RT, Rous Associated Virus (RAV) RT, and Myeloblastosis Associated Virus (MAV) RT. Table 1 provides several examples of retroviral proteins comprising reverse transcriptase domains (e.g., RSV, AMV type 1, AMV type 2, and RAV type 2) (reverse transcriptase domains indicated in bold font).

TABLE 1

| SEQ ID | | |
|---|---|---|
| NO: | Description | Amino Acid Sequence |
| 23 | RSV, pol | TVALHLAIPLKWKPDHTPVWIDQWPLPEGKLVALTQLVEKEL |
| | | QLGHIVPSLSCWNTPVFVIRKASGSYRLLHDLRAVNAKLVPF |
| | | GAVQQGAPVLSALPRGWPLMVLDLKDCFFSIPLAEQDREAF |
| | | AFTLPSVNNQAPARRFQWKVLPQGMTCSPTICQLVVGQVLE |
| | | PLRLKHPSLCMLHYMDDLLLAASSHDGLEAAGEEVISTLER |
| | | AGFTISPDKVQREPGVQYLGYKLGSTYVAPVGLVAEPRIATL |
| | | WDVQKLVGSLQWLRPALGIPPRLMGPFYEQLRGSDPNEAREWN |
| | | LDMKMAWREIVQLSTTAALERWDPALPLEGAVARCEQGAIGV |
| | | LGQGLSTHPRPCLWLFSTQPTKAFTAWLEVLTLLITKLRASAVR |
| | | TFGKEVDILLLPACFREDLPLPEGILLALKGFAGKIRSSDTPSIFDI |
| | | ARPLHVSLKVRVTDHPVPGPTVFTDASSSTHKGVVVWREGPRW |
| | | EIKEIADLGASVQQLEARAVAMALLLWPTAPTNVVTDSAFVAK |
| | | MLLKMGQEGVPSTAAAFILEDALSQRSAMAAVLHVRSHSEVPG |
| | | FFTEGNDVADSKATFQAYPLREAKDLHTALHIGPRALSKACNIS |
| | | MQQAREVVQTCPHCNSAPALEAGVNPRGLGPLQIWQTDFTLEP |
| | | RMAPRSWLAVTVDTASSAIVVTQHGRVTSVAAQHHWATAIAV |
| | | LGRPKAIKTDNGSCFTSKSTREWLARWGIAHTTGIPGNSQGQAM |
| | | VERANRLLKDRIRVLAEGDGFMKRIPTSKQGELLAKAMYALNH |
| | | FERGENTKTPIQKHWRPTVLTEGPPVKIRIETGEWEKGWNVLV |
| | | WGRGYAAVKNRDTDKVIWVPSRKVKPDITQKDEVTKKDEASP |
| | | LFAGISDWIPWEDEQEGLQGETASNKQERPGEDTLAANES |
| 24 | AMV type 1, partial | RATVLTVALHLAIPLKWKPNHTPVWIDQWPLPEGKLVALTQL |
| | pol | VEKELQLGHIEPSLSCWNTPVFVIRKASGSYRLLHDLRAVNA |
| | | KLVPFGAVQQGAPVLSALPRGWPLMVLDLKDCFFSIPLAEQ |
| | | DREAFAFTLPSVNNQAPARRFQWKVLPQGMTCSPTICQLIV |
| | | GQILEPLRLKHPSLRMLHYMDDLLLAASSHDGLEAAGEEVIS |
| | | TLERAGFTISPDKVQREPGVQYLGYKLGSTYVAPVGLVAEPRI |
| | | ATLWDVQKLVGSLQSVRPALGIPPRLMGPFYEQLRGSDPNEARE |
| | | WNLDMKMAWREIVQLSTTAALERWDPALPLEGAVARCEQGAI |
| | | GVLGQGLSTHPRPCLWLFSTQPTKAFTAWLEVLTLLITKLRASA |
| | | VRTFGKEVDILLLPACFREDLPLPEGILLALRGFAGKIRSSDTPSIF |
| | | DIARPLHVSLKVRVTDHPVPGPTVFTDASSSTHKGVVVWREGPR |
| | | WEIKEIADLGASVQQLEARAVAMALLLWPTTPTNVVTDSAFVA |
| | | KMLLKMGQEGVPSTAAAFILEDALSQRSAMAAVLHVRSHSEVP |
| | | GFFTEGNDVADSQATFQAYPLREAKDLHTALHIGPRALSKACNI |
| | | SMQQAREVVQTCPHCNSAPALEAGVNPRGLGPLQIWQTDFTLE |
| | | PRMAPRSWLAVTVDTASSAIVVTQHGRVTSVAAQHHWATAIA |
| | | VLGRPKAIKTDNGSCFTSKSTREWLARWGIAHTTGIPGNSQGQA |
| | | MVERANRLLKDKIRVLAEGDGFMKRIPTSKQGELLAKAMYALN |
| | | HFERGENTKTPIQKHWRPTVLTEGPPVKIRIETGEWEKGWNVLV |
| | | WGRGYAAVKNRDTDKVIWVPSRKVKPDITQKDEVTKKDEASP |
| | | LFAGISDWAPWEGEQEGLQEETASNKQERPGEDTPAANES |
| 25 | AMV type 2, partial | GRATVFTVALHLAIPLKWKPDHTPVWIDQWPLPEGKLVALTQ |
| | pol | LVEKELQLGHIEPSLSCWNTPVFVIRKASGSYRLLHDLRAVN |
| | | AKLVPFGAVQQGAPVLSALPRGWPLMVLDLKDCFFSIPLAE |
| | | QDREAFAFTLPSVNNQAPARRFQWKVLPQGMTCSPTICQLI |
| | | VGQILEPLRLKHPSLRMLHYMDDLLLAASSHDGLEAAGEEV |
| | | ISTLERAGFTISPDKVQKEPGVQYLGYKLGSTYVAPVGLVAEP |
| | | RIATLWDVQKLVGSLQSVRPALGIPPRLMGPFYEQLRGSDPNEA |
| | | REWNLDMKMAWREIVQLSTTAALERWDPALPLEGAVARCEQG |
| | | AIGVLGQGLSTHPRPCLWLFSTQPTKAFTAWLEVLTLLITKLRAS |
| | | AVRTFGKEVDILLLPACFREDLPLPEGILLALRGFAGKIRSSDTPS |
| | | IFDIARPLHVSLKVRVTDHPVPGPTVFTDASSSTHKGVVVWREG |
| | | PRWEIKEIADLGASVQQLEARAVAMALLLWPTTPTNVVTDSAF |
| | | VAKMLLKMGQEGVPSTAAAFILEDALSQRSAMAAVLHVRSHSE |
| | | VPGFFTEGNDVADSQATFQAYPLREAKDLHTALHIGPRALSKAC |
| | | NISMOQAREVVQTCPHCNSAPALEAGVNPRGLGPLQIWQTDFT |
| | | LEPRMAPRSWLAVTVDTASSAIVVTQHGRVTSVAAQHHWATAI |
| | | AVLGRPKAIKTDNGSCFTSKSTREWLARWGIAHTTGIPGNSQGQ |
| | | AMVERANRLLKDKIRVLAEGDGFMKRIPTSKQGELLAKAVYAL |
| | | NHFERGENTKTPIQKHWRPTVLTEGPPVKIRIETGEWEKGWNVL |
| | | VWGRGYAAVKNRDTDKVIWVPSRKVKPDITQKDEVTKRDEAS |
| | | PLFAGISDWAPWEGEQEGLQEETASNKQERPGEDTLAANES |
| 26 | Rous-associated virus | TVALHLAIPLKWKPDHTPVWIDQWPLPEGKLVAVTQLVEKE |
| | type 2, partial RT | LQLGHIEPSLSCWNTPVFVIRKASGSYRLLHDLRAVNAKLVP |
| | | FGAVQQGAPVLSALPRGWPLMVLDLKDCFFSIPLAEQDREA |
| | | FAFTLPSVNNQAPARRFQWKVLPQGMTCSPTICQLVVGQVL |
| | | EPLRLKHPALRMLHYMDDLLLAASSHDGLEAAGKEVIGTLE |
| | | RAGFTISPDKIQREPGVQYLGYKLGSTYVAPVGLVAEPRIATL |
| | | WDVQKLVGSLQWLRPALGIPPRLMGPFYEQLRGSDPNEAREWN |
| | | LDMKMAWREIVQLSTTAALERWDPAQPLEGAVARCEQGAIGV |
| | | LGQGLSTHPRPCLWLFSTQPTKAFTAWLEVLTLLITKLRASAVR |
| | | TFGKEVDILLLPACFREDLPLPEGILLALRGFAGKIRSSDTPSIFDI |
| | | ARPLHVSLKVRVTDHPVPGPTVFTDASSSTHKGVVVWREGPRW |
| | | EIKEIVDLGASVQQLEARAVAMALLLWPTTPTNVVTDSAFVAK |
| | | MLLKMGQEGVPSTAAAFILEDALSQRSAMAAVLHVRSHSEVPG |
| | | FFTEGNDVADSQATFQAYPLREAKDLHTALHIGPRALSKACNIS |
| | | MQQAREVVQTCPHCNSAPALEAGVNPRGLGPLQIWQTDFTLEP |
| | | RMAPRSWLAVTVDTASSAIVVTQHGRVTSVAAQHHWATAIAV |
| | | LGRPKAIKTDNGSCFTSKSTREWLARWGIAHTTGIPGNSQGQAM |
| | | VERANRLLKDKIRVLAEGDGFMKRIPASKQGELLAKAMYALNH |
| | | FERGENTKTPVQKHWRPTVLTEGPPVKIRIETGEWEKGWNVLV |
| | | WGRGYAAVKNRDTDKVIWVPSRKVKPDITQKDEVTKKDEASP |
| | | LFAGSSDWIPWGDEQEGLQEEAASNKQEGPGEDTLAANES |

In some instances, the polymerase domain of a chimeric RT provided herein comprises a polymerase identical to or derived from M-MLV. M-MLV RT (SEQ ID NO: 1) contains a single subunit of ˜75 kDa that comprises a RNA-dependent DNA polymerase domain (SEQ ID NO: 2) (˜510 amino acids in length) and a RNase H domain (SEQ ID NO: 3). In some instances, a non-natural RT provided herein comprises a domain derived from a M-MLV polymerase domain (e.g., SEQ ID NO: 2). In some instances, the domain derived from a M-MLV polymerase domain comprises an active site domain comprising 109 (R), 118 (N), 149 (D), 187 (L), 188 (P), 189 (Q), 190 (G), 221 (Y), 223 (D), and 224 (D) of SEQ ID NO: 2. In some instances, a non-natural RT provided herein comprising a domain derived from a M-MLV polymerase domain comprises at least one mutation at an active site residue, for example, at one of residues 109 (R), 118 (N), 149 (D), 187 (L), 188 (P), 189 (Q), 190 (G), 221 (Y), 223 (D), and 224 (D) of SEQ ID NO: 2. In some embodiments, the mutation at the active site residue is a conservative mutation (e.g., a mutation to an amino acid with similar biochemical and/or physicochemical properties (e.g. charge, hydrophobicity, size, positively charged, negatively charged, etc.)). The conservative mutation of the active site residue of the polymerase domain of a chimeric RT can result in a change to an amino acid having the same or similar properties (e.g., hydrophobic, hydrophilic, polar, non-polar, positively charged, negatively charged, etc.) as the original, unmutated residue. In some embodiments, the conservative mutation of the active site residue results in a change (e.g., increase or decrease) in the activity (e.g., enzymatic activity) of the M-MLV polymerase domain. For example, the conservative mutation in the active site can increase the activity of the domain by improving the binding ability and/or affinity of the polymerase domain to a binding partner. In some embodiments, the mutation at the active site residue can be a non-conservative mutation (e.g., a mutation to an amino acid with dissimilar biochemical and/or physicochemical properties (e.g., charge, hydrophobicity, size, etc.)). The non-conservative mutation in the active site residue of a polymerase domain of a chimeric RT can result in a change to an amino acid having different or dissimilar properties as the unmutated residue (e.g., hydrophobic, hydrophilic, polar, non-polar, etc.). In some embodiments, the non-conservative mutation of the active site residue results in a change (e.g., increase or decrease) in the activity (e.g., enzymatic activity) of the M-MLV polymerase domain. For example, the non-conservative mutation in the active site can decrease the activity of the domain by decreasing the ability of the polymerase domain to bind to a binding partner. The effect on enzymatic activity of certain mutations in the active site can, in some cases, be predicted based on computational modeling and/or empirical observations. However, in some cases, the effect of mutations in the active site may not be accurately predicted. Mutations at active sites can be optimized by various experimental methods, including high-throughput screening of variant libraries.

In some embodiments, a non-natural RT provided herein comprises a polymerase domain derived from a M-MLV polymerase domain (e.g., SEQ ID NO: 2), and the polymerase domain comprises at least one conservative mutation at one of residues 109 (R), 118 (N), 149 (D), 187 (L), 188 (P), 189 (Q), 190 (G), 221 (Y), 223 (D), and 224 (D) of SEQ ID NO: 2. In some embodiments, a non-natural RT provided herein comprises a polymerase domain derived from a M-MLV polymerase domain (e.g., SEQ ID NO: 2), and the polymerase domain comprises at least one non-conservative mutation at one of residues 109 (R), 118 (N), 149 (D), 187 (L), 188 (P), 189 (Q), 190 (G), 221 (Y), 223 (D), and 224 (D) of SEQ ID NO: 2. In certain embodiments, at least one mutation at an active site residue (e.g., 109 (R), 118 (N), 149 (D), 187 (L), 188 (P), 189 (Q), 190 (G), 221 (Y), 223 (D), and 224 (D) of SEQ ID NO: 2) results in increased activity of the domain. In certain embodiments, at least one mutation at an active site residue (e.g., 109 (R), 118 (N), 149 (D), 187 (L), 188 (P), 189 (Q), 190 (G), 221 (Y), 223 (D), and 224 (D) of SEQ ID NO: 2) results in decreased activity of the domain. In some embodiments, the mutation in the polymerase domain is at an amino acid residue other than an active site residue. A mutation at an amino acid residue other than an active site residue can, in some cases, affect the active site (e.g., conformation of the active site) and consequently affect the activity of the polymerase domain.

In some instances, the polymerase domain is an HIV polymerase domain. Human Immunodeficiency Virus-1 (e.g., HIV-1) RT exists as a heterodimer of p66 and p51 subunits in which the smaller subunit (p51, ˜51 kDa) is derived from the larger subunit (p66, ˜66 kDa) by proteolytic cleavage. The larger subunit of the RT heterodimer, p66 (SEQ ID NO: 4), contains the active sites for both of the enzymatic activities of RT (e.g., polymerase and RNase H). A polymerase domain of a non-natural RT provided herein may thus comprise a polymerase domain such as amino acids 1-437 of SEQ ID NO: 4 (SEQ ID NO: 5) or a domain derived from SEQ ID NO: 5. A non-natural RT may also comprise a structure sub-unit, such as a sub-unit derived from p51 of HIV.

Avian Sarcoma-Leukosis Virus (ASLV) RT can also be found as a heterodimer of two subunits, alpha which is ˜62 kDa and beta which is ˜94 kDa. The alpha subunit is derived from the beta subunit by proteolytic cleavage.

### Ribonuclease Domain

A non-natural RT provided herein can comprise an RNase domain that exhibits RNase activity. The RNase can be identical to, or derived from, a naturally-occurring RNase domain. In some embodiments, the RNase domain is structurally similar to a natural RNase. In some cases, the RNase domain does not exhibit RNase activity or exhibits reduced RNase activity. Generally, the RNase activity catalyzes the degradation of RNA into smaller components. The RNase activity can be RNase H activity. The RNase domain may cleave RNA (e.g., mRNA) in a DNA/RNA duplex to produce ssDNA. In some cases, the RNase activity is non-specific. In some cases, the RNase activity is an endonuclease activity; in some cases, it may cleave RNA via a hydrolytic mechanism, sometimes with the aid of an enzyme-bound divalent metal ion. Generally, the RNase domain does not hydrolyze the phosphodiester bonds within single-stranded and double-stranded DNA or RNA.

In some instances, an RNase domain within a non-natural RT provided herein may be derived from a virus (e.g., retrovirus, HIV, HIV1, HIV2), a lentivirus, a bacterium (e.g., an extremophilic or thermophilic bacterium), an archaea (e.g., an extremophilic or thermophilic bacterium), microorganism, eukaryote, or multicellular organism (e.g, human). In some cases, the RNase domain is identical to, or derived from RNase H, RNase H1, RNase HII, or RNase HIII.

The three-dimensional structure of RNase within a non-natural RT provided herein may be identical to, or similar to that of a natural RNase. For example, a RNase domain provided herein may comprise a 5-stranded β-sheet surrounded by a distribution of α-helices. In some cases, the RNase H does not comprise one or more helices (e.g, a C-helix).

An RT provided herein may comprise an RNase derived from an organism with high levels of thermal stability. For example, the RNase may be derived from a bacterial or archaeal RNase that can function at temperatures of at least 30° C. (e.g., at least about 32° C., at least about 34° C., at least about 36° C., at least about 38° C., at least about 40° C., at least about 42° C., at least about 45° C., at least about 50° C., at least about 55° C., at least about 60° C., at least about 65° C., at least about 70° C., at least about 75° C., at least about 80° C., at least about 85° C., or at least about 90° C.). In some cases, the RNase may be derived from a bacterial or archaeal RNase that can function at temperatures of at least 40° C. In some examples, the RNase within an RT provided herein may be derived from a extremophilic (e.g. thermophilic) bacterium (e.g., T. thermophilus, SEQ ID NO: 7) that can grow optimally at temperatures of about 66° C.-68° C. or an extremophilic (e.g. thermophilic) archaeon (e.g., T. litoralis, SEQ ID NO: 8) that can grow optimally at temperatures of about 85° C.-88° C.

In some cases, the RNase domain is derived from an M-MLV RT RNase domain, e.g., (SEQ ID NO: 3). In some cases, the RNase domain is derived from an HIV RT RNase (e.g., SEQ ID NO: 6).

The function or structure of an RNase within an RT provided herein may be reduced or increased, for example by mutation (or genetic modification). A ribonuclease domain with reduced ribonuclease activity may have less than about 95%, less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, less than about 7.5%, less than about 5%, or less than about 2.5% of the ribonuclease activity of the corresponding unmutated ribonuclease, such as the ribonuclease domain of a wild-type viral RT (e.g., M-MLV RT, HIV-1 RT, AMV RT, RSV RT), or the ribonuclease activity of an unmutated cellular RNase H (e.g., bacterial or archaeal RNase H, thermophilic RNase H). In some cases, the chimeric RTs of the disclosure may have at least 50% (e.g., at least 55%, at least 60%, at least 65%, at least 70%, at least 75%, at least 80%, at least 85%, at least 90%, or at least 95%) of the RNase H activity compared to the corresponding wild-type RT and/or un-mutated ribonuclease domain. In some cases, the chimeric RTs of the disclosure may have at least 50% and less than 100% of the RNase H activity compared to the corresponding wild-type RT and/or un-mutated ribonuclease domain. In contrast, in some embodiments, a chimeric RT of the disclosure may not contain a modification or mutation in the RNase H domain and may not contain a modification which reduces RNase H activity.

A ribonuclease domain in a non-natural RT provided herein may comprise one or more truncations. For example, it may be truncated at the N-terminus, e.g., by at least 1 amino acid (e.g., at least 2, 3, 4, 5, 6, 7, 8 amino acids or more than 8 amino acids). In some cases, the ribonuclease domain within a non-natural RT provided herein may be truncated at the C-terminus, e.g., by at least 1 amino acid (e.g., at least 2, 3, 4, 5, 6, 7, 8 amino acids or more than 8 amino acids).

A ribonuclease domain within in a non-natural RT provided herein may comprise one or more mutations compared to the wild-type sequence of the domain. In some cases, the ribonuclease domain comprises at least 1 mutation (e.g., at least 2, 3, 4, 5, 6, 7, 8 mutations or more than 8 mutations). In some cases, the ribonuclease domain may be at least 60% identical (e.g., at least 65%, 70%, 75%, 80%, 85%, 90%, or 95% identical) and less than 100% identical to a wild-type RNase domain.

A non-natural RT provided herein can comprise a thermophilic ribonuclease domain. For example, the thermophilic ribonuclease domain may be identical to or derived from T. thermophilus (SEQ ID NO: 7) or T. litoralis (SEQ ID NO: 8). In specific examples, such ribonuclease domain comprises a truncation or mutation (e.g., SEQ ID NO: 21 and SEQ ID NO: 22).

In some particular examples, the ribonuclease domain derived from a thermophile, e.g., SEQ ID NO: 7 and SEQ ID NO: 8, may have one or more mutations compared to the wild-type sequence of the domain, e.g., SEQ ID NO: 21 and SEQ ID NO: 22. A mutation compared to the wild-type sequence of the domain may be an active site mutation. In some cases, the ribonuclease domain comprises at least 1 mutation (e.g., at least 2, 3, 4, 5, 6, 7, 8 mutations or more than 8 mutations) relative to the wild-type sequence of the domain. In some cases, the ribonuclease domain may be at least 60% identical (e.g., at least 65%, 70%, 75%, 80%, 85%, 90%, or 95% identical) and less than 100% identical to wild-type Tth RNase H (SEQ ID NO: 7) or wild-type Tli RNase H (SEQ ID NO: 8). In some cases, the ribonuclease domain may be at least 50% identical and less than 100% identical to SEQ ID NO: 7 or SEQ ID NO: 8. In some cases, the ribonuclease domain may be at least 50% identical and less than 99.9% identical to SEQ ID NO: 7 or SEQ ID NO: 8; less than 99.8% identical to SEQ ID NO: 7 or SEQ ID NO: 8; less than 99.7% identical to SEQ ID NO: 7 or SEQ ID NO: 8; less than 99.6% identical to SEQ ID NO: 7 or SEQ ID NO: 8; less than 99.5% identical to SEQ ID NO: 7 or SEQ ID NO: 8; less than 99.4% identical to SEQ ID NO: 7 or SEQ ID NO: 8; less than 99.3% identical to SEQ ID NO: 7 or SEQ ID NO: 8; or less than 99% identical to SEQ ID NO: 7 or SEQ ID NO: 8. In some cases, the ribonuclease domain may be at least 75% identical and less than 99.8% identical to wild-type Tth RNase H (SEQ ID NO: 7) or wild-type Tli RNase H (SEQ ID NO: 8).

Mutations such as, for example, substitutions at an active site can be introduced to affect the enzymatic activity of the domain. Homologous proteins that are similar at the sequence level, at the structural level, and/or at the functional level may have similar active sites, for example in terms of 3D structure, amino acid characteristics (e.g., sequence), and other biochemical properties. RNase H's generally have an active site centered on a conserved sequence motif composed of aspartate and glutamate residues, often referred to as the DEDD motif. These residues interact with catalytically required magnesium ions and mutation of these residues and the corresponding homologous residues in related proteins may result in RNases with increased or reduced enzymatic activities. The DEDD motif, can be found in T. thermophilus (SEQ ID NO: 7) at, for example, residues 14 (D), 52 (E), 74 (D) and 139 (D). The DEDD motif, can be found in T. litoralis (SEQ ID NO: 8) at, for example, residues 7 (D), 8 (E), 105 (D) and 135 (D).

In some embodiments, a non-natural RT provided herein comprises a ribonuclease domain derived from T. thermophilus (SEQ ID NO: 7). In some embodiments, a non-natural RT provided herein comprising a domain derived from T. thermophilus comprises at least one mutation at an active site residue, for example, at one of residues 14 (D), 52 (E), 74 (D) and 139 (D) of SEQ ID NO: 7. In some embodiments, the mutation at the active site residue is a conservative mutation (e.g., a mutation to an amino acid with similar biochemical and/or physicochemical properties (e.g., charge, hydrophobicity, size, etc.)). The conservative mutation of the active site residue of the ribonuclease domain of a chimeric RT can result in a change to an amino acid having the same or similar properties (e.g., hydrophobic, hydrophilic, polar, non-polar, positively charged, negatively charged etc.) as the original, unmutated residue. In some embodiments, the conservative mutation of the active site residue results in a change (e.g., increase or decrease) in the activity (e.g., enzymatic activity) of the T. thermophilus ribonuclease domain. For example, the conservative mutation in the active site can increase the activity of the domain by improving the binding ability and/or affinity of the ribonuclease domain to a binding partner. In some embodiments, the mutation at the active site residue is a non-conservative mutation (e.g., a mutation to an amino acid amino acid having different or dissimilar properties as the unmutated residue (e.g., hydrophobic, hydrophilic, polar, non-polar, positively charged, negatively charged, etc.)). In some embodiments, the non-conservative mutation of the active site residue results in a change (e.g., increase or decrease) in the activity (e.g., enzymatic activity) of the ribonuclease domain. For example, the non-conservative mutation in the active site can decrease the activity of the domain by decreasing the ability of the ribonuclease domain to bind a binding partner. The effect on enzymatic activity of certain mutations in the active site can, in some cases, be predicted based on computational modeling and/or empirical observations. However, in some cases, the effect of mutations in the active site may not be accurately predicted. Mutations at active sites can be optimized by various experimental methods, including high-throughput screening of variant libraries.

In some embodiments, a non-natural RT provided herein comprises a ribonuclease domain derived from a T. thermophilus RNase H domain (e.g., SEQ ID NO: 7), and the ribonuclease domain comprises at least one conservative mutation at one of residues 14 (D), 52 (E), 74 (D) and 139 (D) of SEQ ID NO: 7. In some embodiments, a non-natural RT provided herein comprises a ribonuclease domain derived from a T. thermophilus RNase H domain (e.g., SEQ ID NO: 7), and the ribonuclease domain comprises at least one non-conservative mutation at one of residues 14 (D), 52 (E), 74 (D) and 139 (D) of SEQ ID NO: 7. In certain embodiments, at least one mutation at an active site residue (e.g., 14 (D), 52 (E), 74 (D) and 139 (D) of SEQ ID NO: 7) results in increased ribonuclease activity of the domain. In certain embodiments, at least one mutation at an active site residue (e.g., 14 (D), 52 (E), 74 (D) and 139 (D) of SEQ ID NO: 7) results in decreased ribonuclease activity of the domain. In some embodiments, the mutation in the ribonuclease domain is at an amino acid residue other than an active site residue. A mutation at an amino acid residue other than an active site residue can, in some cases, affect the active site (e.g., conformation of the active site) and consequently affect the activity of the ribonuclease domain. In some embodiments, an amino acid mutation from the amino acid Asp (D) to the amino acid Asn (N) at residue 14 of SEQ ID NO: 7 (SEQ ID NO: 21), which may be an active site residue, decreases and/or eliminates the enzymatic activity of this domain. Such modification may minimize degradation of RNA, for example during first strand synthesis, thereby possibly increasing cDNA yield.

In some embodiments, a non-natural RT provided herein comprises a ribonuclease domain derived from T. litoralis (SEQ ID NO: 8). In some embodiments, a non-natural RT provided herein comprising a domain derived from T. litoralis comprises at least one mutation at an active site residue, for example at one of residues 7 (D), 8 (E), 105 (D) and 135 (D) of SEQ ID NO: 8. In some embodiments, the mutation at the active site residue is a conservative mutation (e.g., a mutation to an amino acid with similar biochemical and/or physicochemical properties (e.g., charge, hydrophobicity, size, etc.)). The conservative mutation in the active site residue of the ribonuclease domain of a chimeric RT can result in a change to an amino acid having the same or similar properties (e.g., hydrophobic, hydrophilic, polar, non-polar, positively charged, negatively charged, etc.) as the original, unmutated residue. In some embodiments, the conservative mutation of the active site residues results in a change (e.g., increase or decrease) in the activity (e.g., enzymatic activity) of the T. litoralis ribonuclease domain. For example, the conservative mutation in the active site can increase the activity of the domain by improving the binding ability and/or affinity of the ribonuclease domain to a binding partner. In some embodiments, the mutation at the active site residue can be a non-conservative mutation (e.g., a mutation to an amino acid with dissimilar biochemical and/or physicochemical properties (e.g., charge, hydrophobicity, size, etc.)). The non-conservation mutation of the active site residue of a ribonuclease domain of a chimeric RT can result in a change to an amino acid having different or dissimilar properties as the unmutated residue (e.g., hydrophobic, hydrophilic, polar, non-polar, positively charged, negatively charged, etc.). In some embodiments, the non-conservative mutation of the active site residue results in a change (e.g., increase or decrease) in the activity (e.g., enzymatic activity) of the ribonuclease domain. For example, the non-conservative mutation in the active site can decrease the activity of the domain by decreasing the ability of the ribonuclease domain to bind a binding partner. The effect on enzymatic activity of certain mutations in the active site can, in some cases, be predicted based on computational modeling and/or empirical observations. However, in some cases, the effect of mutations in the active site may not be accurately predicted. Mutations at active sites can be optimized by various experimental methods, including high-throughput screening of variant libraries.

In some embodiments, a non-natural RT provided herein comprises a ribonuclease domain derived from a T. litoralis RNase H domain (e.g., SEQ ID NO: 8), and the ribonuclease domain comprises at least one conservative mutation at one of residues 7 (D), 8 (E), 105 (D) and 135 (D) of SEQ ID NO: 8. In some embodiments, a non-natural RT provided herein comprises a ribonuclease domain derived from a T. litoralis RNase H domain (e.g., SEQ ID NO: 8), and the ribonuclease domain comprises at least one non-conservative mutation at one of residues 7 (D), 8 (E), 105 (D) and 135 (D) of SEQ ID NO: 8. In certain embodiments, at least one mutation at an active site residue (e.g., 7 (D), 8 (E), 105 (D) and 135 (D) of SEQ ID NO: 8) results in increased ribonuclease activity of the domain. In certain embodiments, at least one mutation at an active site residue (e.g., 7 (D), 8 (E), 105 (D) and 135 (D) of SEQ ID NO: 8) results in decreased ribonuclease activity of the domain. In some embodiments, the mutation in the ribonuclease domain is at an amino acid residue other than an active site residue. A mutation at an amino acid residue other than an active site residue can, for example, affect the active site (e.g., conformation of the active site) and consequently affect the activity of the ribonuclease domain. In some embodiments, the domain comprises two amino acid mutations—from Asp (D) to Asn (N) at residue 7 and Glu (E) to Gin (Q) at residue 8 of SEQ ID NO: 8 (SEQ ID NO: 22). Two or more mutations can result in decreased enzymatic activity (e.g., ribonuclease activity) of the ribonuclease domain. Such modification may minimize RNA degradation.

TABLE 2
Amino acid sequences

| SEQ | | |
|---|---|---|
| ID NO: | Description | Amino Acid Sequence |
| 1 | wt M-MLV RT (pol | LNIEDEHRLHETSKEPDVSLGSTWLSDFPQAWAETGG |
| | domain and RNase H | MGLAVRQAPLIIPLKATSTPVSIKQYPMSQEARLGIKP |
| | domain) | HIQRLLDQGILVPCQSPWNTPLLPVKKPGTNDYRPVQ |
| | | DLREVNKRVEDIHPTVPNPYNLLSGLPPSHQWYTVLD |
| | | LKDAFFCLRLHPTSQPLFAFEWRDPEMGISGQLTWTR |
| | | LPQGFKNSPTLFDEALHRDLADFRIQHPDLILLQYVD |
| | | DLLLAATSELDCQQGTRALLQTLGNLGYRASAKKAQ |
| | | ICQKQVKYLGYLLKEGQRWLTEARKETVMGQPTPKT |
| | | PRQLREFLGTAGFCRLWIPGFAEMAAPLYPLTKTGTL |
| | | FNWGPDQQKAYQEIKQALLTAPALGLPDLTKPFELFV |
| | | DEKQGYAKGVLTQKLGPWRRPVAYLSKKLDPVAAG |
| | | WPPCLRMVAAIAVLTKDAGKLTMGQPLVILAPHAVE |
| | | ALVKQPPDRWLSNARMTHYQALLLDTDRVQFGPVV |
| | | ALNPATLLPLPEEGLQHNCLDILAEAHGTRPDLTDQP |
| | | LPDADHTWYTDGSSLLQEGQRKAGAAVTTETEVI |
| | | WAKALPAGTSAQRAELIALTQALKMAEGKKLNV |
| | | YTDSRYAFATAHIHGEIYRRRGLLTSEGKEIKNKD |
| | | EILALLKALFLPKRLSIIHCPGHQKGHSAEARGNR |
| | | MADQAARKAAITETPDTSTLLI |
| 2 | wt M-MLV pol | LNIEDEHRLHETSKEPDVSLGSTWLSDFPQAWAETGG |
| | domain | MGLAVRQAPLIIPLKATSTPVSIKQYPMSQEARLGIKPH |
| | | IQRLLDQGILVPCQSPWNTPLLPVKKPGTNDYRPVQDL |
| | | REVNKRVEDIHPTVPNPYNLLSGLPPSHQWYTVLDLK |
| | | DAFFCLRLHPTSQPLFAFEWRDPEMGISGQLTWTRLPQ |
| | | GFKNSPTLFDEALHRDLADFRIQHPDLILLQYVDDLLL |
| | | AATSELDCQQGTRALLQTLGNLGYRASAKKAQICQKQ |
| | | VKYLGYLLKEGQRWLTEARKETVMGQPTPKTPRQLR |
| | | EFLGTAGFCRLWIPGFAEMAAPLYPLTKTGTLFNWGP |
| | | DQQKAYQEIKQALLTAPALGLPDLTKPFELFVDEKQG |
| | | YAKGVLTQKLGPWRRPVAYLSKKLDPVAAGWPPCLR |
| | | MVAAIAVLTKDAGKLTMGQPLVILAPHAVEALVKQPP |
| | | DRWLSNARMTHYQALLLDTDRVQFGPVVALNPATLL |
| | | PLPEEGLQHNCLDILAEAHGTRPDLTD |
| 3 | wt M-MLV RNase H | QPLPDADHTWYTDGSSLLQEGQRKAGAAVTTETEVIW |
| | domain | AKALPAGTSAQRAELIALTQALKMAEGKKLNVYTDSR |
| | | YAFATAHIHGEIYRRRGLLTSEGKEIKNKDEILALLKAL |
| | | FLPKRLSIIHCPGHQKGHSAEARGNRMADQAARKAAIT |
| | | ETPDTSTLLI |
| 4 | wt HIV-1 RT (pol | NFPISPIETVPVKLKPGMDGPKVKQWPLTEEKIKALVEI |
| | domain and RNase H | CTEMEKEGKISKIGPENPYNTPVFAIKKKDSTKWRKLV |
| | domain) | DFRELNKRTQDFWEVQLGIPHPAGLKKKKSVTVLDVG |
| | | DAYFSVPLDEDFRKYTAFTIPSINNETPGIRYQYNVLPQ |
| | | GWKGSPAIFQSSMTKILEPFRKQNPDIVIYQYMDDLYV |
| | | GSDLEIGQHRTKIEELRQHLLRWGLTTPDKKHQKEPPF |
| | | LWMGYELHPDKWTVQPIVLPEKDSWTVNDIQKLVGK |
| | | LNWASQIYPGIKVRQLCKLLRGTKALTEVIPLTEEAELE |
| | | LAENREILKEPVHGVYYDPSKDLIAEIQKQGQGQWTY |
| | | QIYQEPFKNLKTGKYARMRGAHTNDVKQLTEAVQKIT |
| | | TESIVIWGKTPKFKLPIQKETWETWWTEYWQATWIPE |
| | | WEFVNTPPLVKLWYQLEKEPIVGAETFYVDGAANRE |
| | | TKLGKAGYVTNRGRQKVVTLTDTTNQKTELQAIYL |
| | | ALQDSGLEVNIVTDSQYALGIIQAQPDQSESELVNQI |
| | | IEQLIKKEKVYLAWVPAHKGIGGNEQVDKLVSAGI |
| | | RKVL |
| 5 | wt HIV-1 pol domain | NFPISPIETVPVKLKPGMDGPKVKQWPLTEEKIKALVEI |
| | | CTEMEKEGKISKIGPENPYNTPVFAIKKKDSTKWRKLV |
| | | DFRELNKRTQDFWEVQLGIPHPAGLKKKKSVTVLDVG |
| | | DAYFSVPLDEDFRKYTAFTIPSINNETPGIRYQYNVLPQ |
| | | GWKGSPAIFQSSMTKILEPFRKQNPDIVIYQYMDDLYV |
| | | GSDLEIGQHRTKIEELRQHLLRWGLTTPDKKHQKEPPF |
| | | LWMGYELHPDKWTVQPIVLPEKDSWTVNDIQKLVGK |
| | | LNWASQIYPGIKVRQLCKLLRGTKALTEVIPLTEEAELE |
| | | LAENREILKEPVHGVYYDPSKDLIAEIQKQGQGQWTY |
| | | QIYQEPFKNLKTGKYARMRGAHTNDVKQLTEAVQKIT |
| | | TESIVIWGKTPKFKLPIQKETWETWWTEYWQATWIPE |
| | | WEFVNTPPLVKLWYQLEKEPIV |
| 6 | wt HIV-1 RNase H | GAETFYVDGAANRETKLGKAGYVTNRGRQKVVTLTD |
| | domain | TTNQKTELQAIYLALQDSGLEVNIVTDSQYALGIIQAQP |
| | | DQSESELVNQIIEQLIKKEKVYLAWVPAHKGIGGNEQV |
| | | DKLVSAGIRKVL |
| 7 | wt Tth RNase H | MNPSPRKRVALFTDGACLGNPGPGGWAALLRFHAHE |
| | | KLLSGGEACTTNNRMELKAAIEGLKALKEPCEVDLYT |
| | | DSHYLKKAFTEGWLEGWRKRGWRTAEGKPVKNRDL |
| | | WEALLLAMAPHRVRFHFVKGHTGHPENERVDREARR |
| | | QAQSQAKTPCPPRAPTLFHEEA |
| 8 | wt Tli RNase H II | MNLGGIDEAGRGPVIGPLVIAAVVVDESRMQELEALG |
| | | VKDSKKLTPKRREELFEEIVQIVDDHVIIQLSPEEIDGRD |
| | | GTMNELEIENFAKALNSLKVKPDVLYIDAADVKEKRF |
| | | GDIIGERLSFSPKIIAEHKADSKYIPVAAASILAKVTRDR |
| | | AIEKLKELYGEIGSGYPSDPNTRRFLEEYYKAHGEFPPI |
| | | VRKSWKTLRKIEEKLKAKKTQPTILDFLKKP |
| 21 | Tth RNase H- | MNPSPRKRVALFTNGACLGNPGPGGWAALLRFHAHE |
| | | KLLSGGEACTTNNRMELKAAIEGLKALKEPCEVDLYT |
| | | DSHYLKKAFTEGWLEGWRKRGWRTAEGKPVKNRDL |
| | | WEALLLAMAPHRVRFHFVKGHTGHPENERVDREARR |
| | | QAQSQAKTPCPPRAPTLFHEEA |
| 22 | Tli RNase H II- | MNLGGINQAGRGPVIGPLVIAAVVVDESRMQELEALG |
| | | VKDSKKLTPKRREELFEEIVQIVDDHVIIQLSPEEIDGRD |
| | | GTMNELEIENFAKALNSLKVKPDVLYIDAADVKEKRF |
| | | GDIIGERLSFSPKIIAEHKADSKYIPVAAASILAKVTRDR |
| | | AIEKLKELYGEIGSGYPSDPNTRRFLEEYYKAHGEFPPI |
| | | VRKSWKTLRKIEEKLKAKKTQPTILDFLKKP |
| 9 | wt M-MLV pol | LNIEDEHRLHETSKEPDVSLGSTWLSDFPQAWAETGG |
| | domain-Tth RNase | MGLAVRQAPLIIPLKATSTPVSIKQYPMSQEARLGIKPH |
| | H⁻ | IQRLLDQGILVPCQSPWNTPLLPVKKPGTNDYRPVQDL |
| | (D10N in active site) | REVNKRVEDIHPTVPNPYNLLSGLPPSHQWYTVLDLK |
| | | DAFFCLRLHPTSQPLFAFEWRDPEMGISGQLTWTRLPQ |
| | | GFKNSPTLFDEALHRDLADFRIQHPDLILLQYVDDLLL |
| | | AATSELDCQQGTRALLQTLGNLGYRASAKKAQICQKQ |
| | | VKYLGYLLKEGQRWLTEARKETVMGQPTPKTPRQLR |
| | | EFLGTAGFCRLWIPGFAEMAAPLYPLTKTGTLFNWGP |
| | | DQQKAYQEIKQALLTAPALGLPDLTKPFELFVDEKQG |
| | | YAKGVLTQKLGPWRRPVAYLSKKLDPVAAGWPPCLR |
| | | MVAAIAVLTKDAGKLTMGQPLVILAPHAVEALVKQPP |
| | | DRWLSNARMTHYQALLLDTDRVQFGPVVALNPATLL |
| | | PLPEEGLQHNCLDILAEAHGTRPDLTDQPLPDNPSPRK |
| | | RVALFTNGACLGNPGPGGWAALLRFHAHEKLLSG |
| | | GEACTTNNRMELKAAIEGLKALKEPCEVDLYTDSH |
| | | YLKKAFTEGWLEGWRKRGWRTAEGKPVKNRDLW |
| | | EALLLAMAPHRVRFHFVKGHTGHPENERVDREAR |
| | | RQAQSQAKTPCPPRAPTLFHEEA |
| 10 | wt M-MLV pol | LNIEDEHRLHETSKEPDVSLGSTWLSDFPQAWAETGG |
| | domain-Tli Rnase H | MGLAVRQAPLIIPLKATSTPVSIKQYPMSQEARLGIKPH |
| | II⁻ | IQRLLDQGILVPCQSPWNTPLLPVKKPGTNDYRPVQDL |
| | | REVNKRVEDIHPTVPNPYNLLSGLPPSHQWYTVLDLK |
| | | DAFFCLRLHPTSQPLFAFEWRDPEMGISGQLTWTRLPQ |
| | | GFKNSPTLFDEALHRDLADFRIQHPDLILLQYVDDLLL |
| | | AATSELDCQQGTRALLQTLGNLGYRASAKKAQICQKQ |
| | | VKYLGYLLKEGQRWLTEARKETVMGQPTPKTPRQLR |
| | | EFLGTAGFCRLWIPGFAEMAAPLYPLTKTGTLFNWGP |
| | | DQQKAYQEIKQALLTAPALGLPDLTKPFELFVDEKQG |
| | | YAKGVLTQKLGPWRRPVAYLSKKLDPVAAGWPPCLR |
| | | MVAAIAVLTKDAGKLTMGQPLVILAPHAVEALVKQPP |
| | | DRWLSNARMTHYQALLLDTDRVQFGPVVALNPATLL |
| | | PLPEEGLQHNCLDILAEAHGTRPDLTDQPLPDKLGGIN |
| | | QAGRGPVIGPLVIAAVVVDESRMQELEALGVKDSK |
| | | KLTPKRREELFEEIVQIVDDHVIIQLSPEEIDGRDGT |
| | | MNELEIENFAKALNSLKVKPDVLYIDAADVKEKRF |
| | | GDIIGERLSFSPKIIAEHKADSKYIPVAAASILAKVTR |
| | | DRAIEKLKELYGEIGSGYPSDPNTRRFLEEYYKAHG |
| | | EFPPIVRKSWKTLRKIEEKLKAKKTQPTILDFLKKP |
| 11 | wt HIV-1 pol domain- | NFPISPIETVPVKLKPGMDGPKVKQWPLTEEKIKALVEI |
| | Tth Rnase H⁻ | CTEMEKEGKISKIGPENPYNTPVFAIKKKDSTKWRKLV |
| | | DFRELNKRTQDFWEVQLGIPHPAGLKKKKSVTVLDVG |
| | | DAYFSVPLDEDFRKYTAFTIPSINNETPGIRYQYNVLPQ |
| | | GWKGSPAIFQSSMTKILEPFRKQNPDIVIYQYMDDLYV |
| | | GSDLEIGQHRTKIEELRQHLLRWGLTTPDKKHQKEPPF |
| | | LWMGYELHPDKWTVQPIVLPEKDSWTVNDIQKLVGK |
| | | LNWASQIYPGIKVRQLCKLLRGTKALTEVIPLTEEAELE |
| | | LAENREILKEPVHGVYYDPSKDLIAEIQKQGQGQWTY |
| | | QIYQEPFKNLKTGKYARMRGAHTNDVKQLTEAVQKIT |
| | | TESIVIWGKTPKFKLPIQKETWETWWTEYWQATWIPE |
| | | WEFVNTPPLVKLWYQLEKEPIVNPSPRKRVALFTNGA |
| | | CLGNPGPGGWAALLRFHAHEKLLSGGEACTTNNR |
| | | MELKAAIEGLKALKEPCEVDLYTDSHYLKKAFTEG |
| | | WLEGWRKRGWRTAEGKPVKNRDLWEALLLAMAP |
| | | HRVRFHFVKGHTGHPENERVDREARRQAQSQAKT |
| | | PCPPRAPTLFHEEA |
| 12 | wt HIV-1 pol domain- | NFPISPIETVPVKLKPGMDGPKVKQWPLTEEKIKALVEI |
| | Tli Rnase H II⁻ | CTEMEKEGKISKIGPENPYNTPVFAIKKKDSTKWRKLV |
| | | DFRELNKRTQDFWEVQLGIPHPAGLKKKKSVTVLDVG |
| | | DAYFSVPLDEDFRKYTAFTIPSINNETPGIRYQYNVLPQ |
| | | GWKGSPAIFQSSMTKILEPFRKQNPDIVIYQYMDDLYV |
| | | GSDLEIGQHRTKIEELRQHLLRWGLTTPDKKHQKEPPF |
| | | LWMGYELHPDKWTVQPIVLPEKDSWTVNDIQKLVGK |
| | | LNWASQIYPGIKVRQLCKLLRGTKALTEVIPLTEEAELE |
| | | LAENREILKEPVHGVYYDPSKDLIAEIQKQGQGQWTY |
| | | QIYQEPFKNLKTGKYARMRGAHTNDVKQLTEAVQKIT |
| | | TESIVIWGKTPKFKLPIQKETWETWWTEYWQATWIPE |
| | | WEFVNTPPLVKLWYQLEKEPIVKLGGINQAGRGPVIG |
| | | PLVIAAVVVDESRMQELEALGVKDSKKLTPKRREE |
| | | LFEEIVQIVDDHVIIQLSPEEIDGRDGTMNELEIENFA |
| | | KALNSLKVKPDVLYIDAADVKEKRFGDIIGERLSFSP |
| | | KIIAEHKADSKYIPVAAASILAKVTRDRAIEKLKELY |
| | | GEIGSGYPSDPNTRRFLEEYYKAHGEFPPIVRKSWK |
| | | TLRKIEEKLKAKKTQPTILDFLKKP |

### Linkers

The domains of a chimeric protein, for example the polymerase domain and the ribonuclease domain of a chimeric reverse transcriptase, can be joined by a linker. The domains may be covalently linked or non-covalently linked. As used herein, the term “linker” refers to a molecule that joins at least two other molecules, either covalently or non-covalently, e.g., through hydrogen bonds, ionic or van der Waals interactions, e.g., a nucleic acid molecule that hybridizes to one complementary sequence at the 5′ end and to another complementary sequence at the 3′ end, thus joining two non-complementary sequences. A linker can connect a first polypeptide with at least a second polypeptide. The linker may be a peptide linker or a chemical linker.

Linkers may be of 3 general categories—flexible linkers, rigid linkers, and in vivo cleavable linkers. In addition to linking functional domains together (e.g., flexible and rigid linkers) or releasing free functional domains in vivo (e.g., cleavable linkers), linkers may offer other advantages (e.g., improving biological activity, increasing expression yield, and achieving desirable pharmacokinetic profiles). “Peptide linkers” generally refer to an amino acid sequence or peptide that connects a first polypeptide with a second polypeptide. A peptide linker may be a synthetic sequence (e.g., not naturally occurring in the native polypeptide or protein) or may be a linker sequence native to the protein. The peptide linker can be connected to the first polypeptide and to the second polypeptide by peptide bonds. A peptide linker can be of any suitable length. For example, a peptide linker may be between about 1 and 100 amino acids in length (e.g., between about 10 and 90, about 20 and 80, about 30 and 70, or about 40 and 60 amino acids in length). A peptide linker can be at least 100 amino acids in length (e.g., at least 125, 150, 175, 200, 300, 400, 500 amino acids or longer).

A flexible linker, e.g., a flexible peptide linker, may be used to join domains requiring a certain degree of movement or interaction. For example, a peptide linker may comprise predominantly glycine (G) and serine (S) amino acids, e.g., (GGS)ₙ or (GSG)ₙ where n represents any suitable number of repeats. Glycine and serine are relatively small, non-polar amino acids that have shown little interaction with linked proteins and can lack secondary structure, thereby having minimal or no effect on the structure and the function of the linked proteins. In some cases, rigid linkers may be preferred to provide desired orientations and separation of the linked domains, maintaining the independent functions of each peptide. Rigid linkers may form secondary structure, e.g., alpha helices. For example, a linker comprising primarily proline residues or the sequence (EAAAK), where n represents any suitable number of repeats, may be used to provide a more rigid linker compared to glycine and serine-based linkers. In some cases, a cleavable linker may be preferred to release the functional domains in vivo. The in vivo cleavage of the linkers in fusion polypeptides may be carried out by proteases that are expressed in vivo under pathological conditions (e.g., cancer or inflammation), in specific cells or tissues, or constrained within certain cellular compartments.

### Properties of Non-Natural RTs

A non-natural RT provided herein may be thermally stable at elevated temperatures (e.g., temperatures of at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., at least about 40° C., at least about 45° C., at least about 50° C., at least about 55° C., at least about 60° C., at least about 65° C., at least about 70° C., at least about 75° C., at least about 80° C., at least about 85° C., at least about 90° C., at least about 95° C., or higher). In some cases, a non-natural RT provided herein may be thermally stable at, for example, a temperature between 25° C. and 75° C., between 45° C. and 70° C., between 55° C. and 75° C., between 60° C. and 65° C., or between 60° C. and 70° C. In some cases, the non-natural RTs may retain a certain amount of RNA-dependent DNA polymerase specific activity at elevated temperatures or after incubation at an elevated temperature for at least about 1 minute, at least about 2 minutes, at least about 3 minutes, at least about 4 minutes, at least about 5 minutes, at least about 10 minutes, at least about 15 minutes, at least about 20 minutes, at least about 25 minutes, at least about 30 minutes, at least about 45 minutes, at least about an hour, at least about a day, at least about 2 days, at least about 3 days, at least about 4 days, at least about 5 days, at least about 6 days, at least about 1 week, at least about 2 weeks, at least about 3 weeks, at least about 1 month, at least about 2 months, at least about 3, at least about 4 months, at least about 5 months, at least about 6 months or longer. In some cases, such time period is between 30 minutes and 6 months, 30 minutes and 6 days, 30 minutes and 60 minutes, or other timeframe.

In some cases, a non-natural RT provided herein may retain at least about 5%, 10%, 15%, 20%, 25%, 30%, 35%, 40%, 45%, 50%, 55%, 60%, 65%, 70%, 75%, 80%, 85%, 90%, 95% or greater than 95% specific activity after incubation at an elevated temperature (e.g., temperatures of at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., at least about 40° C., at least about 45° C., at least about 50° C., at least about 55° C., at least about 60° C., at least about 65° C., at least about 70° C., at least about 75° C., at least about 80° C., at least about 85° C., or at least about 90° C.). In some cases, a non-natural RT provided herein may retain at least 50%-100% after incubation at an elevated temperature, e.g., a temperature between 25° C. and 90° C. or between 35° C. and 80° C. or other elevated temperature. In some cases, a non-natural RT provided herein may retain at least about 5%, 10%, 15%, 20%, 25%, 30%, 35%, 40%, 45% 50%, 55%, 60%, 65%, 70%, 75%, 80%, 85%, 90%, 95% or greater than 95% specific activity after incubation at an elevated temperature for at least about 1 minute, at least about 2 minutes, at least about 3 minutes, at least about 4 minutes, at least about 5 minutes, at least about 10 minutes, at least about 15 minutes, at least about 20 minutes, at least about 25 minutes, at least about 30 minutes, at least about 45 minutes, at least about an hour, at least about a day, at least about 2 days, at least about 3 days, at least about 4 days, at least about 5 days, at least about 6 days, at least about 1 week, at least about 2 weeks, at least about 3 weeks, at least about 1 month, at least about 2 months, at least about 3, at least about 4 months, at least about 5 months, at least about 6 months, or longer. In some cases, the percentage of specific activity retained (e.g., 50%, 75%, etc.) is retained between about 10 minutes and about 30 minutes, between 10 minutes and 60 minutes, between 10 minutes and 6 hours, between 10 minutes and 6 months, or other timeframe. In some cases, a non-natural RT provided herein retains at least 25% specific activity after incubation at a temperature of at least about 50° C. for at least 15 minutes. In some cases, a non-natural RT provided herein retains at least 25% specific activity after incubation at a temperature of at least about 50° C. for at least 30 minutes. In some cases, a non-natural RT provided herein retains at least 25% specific activity after incubation at a temperature of at least about 50° C. for at least 45 minutes. In some cases, a non-natural RT provided herein retains at least 25% specific activity after incubation at a temperature of at least about 50° C. for at least 1 hour. In some cases, a non-natural RT provided herein retains at least 25% specific activity after incubation at a temperature of at least about 50° C. for at least 2 hours. In some cases, a non-natural RT provided herein retains at least 25% specific activity after incubation at a temperature of at least about 50° C. for at least 3 hours. In some cases, a non-natural RT provided herein retains at least 25% specific activity after incubation at a temperature of at least about 50° C. for at least 6 hours. In some cases, a non-natural RT provided herein retains at least 50% specific activity after incubation at a temperature of at least about 50° C. for at least 15 minutes. In some cases, a non-natural RT provided herein retains at least 50% specific activity after incubation at a temperature of at least about 50° C. for at least 30 minutes. In some cases, a non-natural RT provided herein retains at least 50% specific activity after incubation at a temperature of at least about 50° C. for at least 45 minutes. In some cases, a non-natural RT provided herein retains at least 50% specific activity after incubation at a temperature of at least about 50° C. for at least 1 hour. In some cases, a non-natural RT provided herein retains at least 50% specific activity after incubation at a temperature of at least about 50° C. for at least 2 hours. In some cases, a non-natural RT provided herein retains at least 50% specific activity after incubation at a temperature of at least about 50° C. for at least 3 hours. In some cases, a non-natural RT provided herein retains at least 50% specific activity after incubation at a temperature of at least about 50° C. for at least 6 hours. In some cases, a non-natural RT provided herein retains at least 75% specific activity after incubation at a temperature of at least about 50° C. for at least 15 minutes. In some cases, a non-natural RT provided herein retains at least 75% specific activity after incubation at a temperature of at least about 50° C. for at least 30 minutes. In some cases, a non-natural RT provided herein retains at least 75% specific activity after incubation at a temperature of at least about 50° C. for at least 45 minutes. In some cases, a non-natural RT provided herein retains at least 75% specific activity after incubation at a temperature of at least about 50° C. for at least 1 hour. In some cases, a non-natural RT provided herein retains at least 75% specific activity after incubation at a temperature of at least about 50° C. for at least 2 hours. In some cases, a non-natural RT provided herein retains at least 75% specific activity after incubation at a temperature of at least about 50° C. for at least 3 hours. In some cases, a non-natural RT provided herein retains at least 75% specific activity after incubation at a temperature of at least about 50° C. for at least 6 hours. In some cases, a non-natural RT provided herein retains at least 25% specific activity after incubation at a temperature of at least about 55° C. for at least 15 minutes. In some cases, a non-natural RT provided herein retains at least 25% specific activity after incubation at a temperature of at least about 55° C. for at least 30 minutes. In some cases, a non-natural RT provided herein retains at least 25% specific activity after incubation at a temperature of at least about 55° C. for at least 45 minutes. In some cases, a non-natural RT provided herein retains at least 25% specific activity after incubation at a temperature of at least about 55° C. for at least 1 hour. In some cases, a non-natural RT provided herein retains at least 25% specific activity after incubation at a temperature of at least about 55° C. for at least 2 hours. In some cases, a non-natural RT provided herein retains at least 25% specific activity after incubation at a temperature of at least about 55° C. for at least 3 hours. In some cases, a non-natural RT provided herein retains at least 25% specific activity after incubation at a temperature of at least about 55° C. for at least 6 hours. In some cases, a non-natural RT provided herein retains at least 50% specific activity after incubation at a temperature of at least about 55° C. for at least 15 minutes. In some cases, a non-natural RT provided herein retains at least 50% specific activity after incubation at a temperature of at least about 55° C. for at least 30 minutes. In some cases, a non-natural RT provided herein retains at least 50% specific activity after incubation at a temperature of at least about 55° C. for at least 45 minutes. In some cases, a non-natural RT provided herein retains at least 50% specific activity after incubation at a temperature of at least about 55° C. for at least 1 hour. In some cases, a non-natural RT provided herein retains at least 50% specific activity after incubation at a temperature of at least about 55° C. for at least 2 hours. In some cases, a non-natural RT provided herein retains at least 50% specific activity after incubation at a temperature of at least about 55° C. for at least 3 hours. In some cases, a non-natural RT provided herein retains at least 50% specific activity after incubation at a temperature of at least about 55° C. for at least 6 hours. In some cases, a non-natural RT provided herein retains at least 75% specific activity after incubation at a temperature of at least about 55° C. for at least 15 minutes. In some cases, a non-natural RT provided herein retains at least 75% specific activity after incubation at a temperature of at least about 55° C. for at least 30 minutes. In some cases, a non-natural RT provided herein retains at least 75% specific activity after incubation at a temperature of at least about 55° C. for at least 45 minutes. In some cases, a non-natural RT provided herein retains at least 75% specific activity after incubation at a temperature of at least about 55° C. for at least 1 hour. In some cases, a non-natural RT provided herein retains at least 75% specific activity after incubation at a temperature of at least about 55° C. for at least 2 hours. In some cases, a non-natural RT provided herein retains at least 75% specific activity after incubation at a temperature of at least about 55° C. for at least 3 hours. In some cases, a non-natural RT provided herein retains at least 75% specific activity after incubation at a temperature of at least about 55° C. for at least 6 hours. In some cases, a non-natural RT provided herein retains at least 25% specific activity after incubation at a temperature of at least about 60° C. for at least 15 minutes. In some cases, a non-natural RT provided herein retains at least 25% specific activity after incubation at a temperature of at least about 60° C. for at least 30 minutes. In some cases, a non-natural RT provided herein retains at least 25% specific activity after incubation at a temperature of at least about 60° C. for at least 45 minutes. In some cases, a non-natural RT provided herein retains at least 25% specific activity after incubation at a temperature of at least about 60° C. for at least 1 hour. In some cases, a non-natural RT provided herein retains at least 25% specific activity after incubation at a temperature of at least about 60° C. for at least 2 hours. In some cases, a non-natural RT provided herein retains at least 25% specific activity after incubation at a temperature of at least about 60° C. for at least 3 hours. In some cases, a non-natural RT provided herein retains at least 25% specific activity after incubation at a temperature of at least about 60° C. for at least 6 hours. In some cases, a non-natural RT provided herein retains at least 50% specific activity after incubation at a temperature of at least about 60° C. for at least 15 minutes. In some cases, a non-natural RT provided herein retains at least 50% specific activity after incubation at a temperature of at least about 60° C. for at least 30 minutes. In some cases, a non-natural RT provided herein retains at least 50% specific activity after incubation at a temperature of at least about 60° C. for at least 45 minutes. In some cases, a non-natural RT provided herein retains at least 50% specific activity after incubation at a temperature of at least about 60° C. for at least 1 hour. In some cases, a non-natural RT provided herein retains at least 50% specific activity after incubation at a temperature of at least about 60° C. for at least 2 hours. In some cases, a non-natural RT provided herein retains at least 50% specific activity after incubation at a temperature of at least about 60° C. for at least 3 hours. In some cases, a non-natural RT provided herein retains at least 50% specific activity after incubation at a temperature of at least about 60° C. for at least 6 hours. In some cases, a non-natural RT provided herein retains at least 75% specific activity after incubation at a temperature of at least about 60° C. for at least 15 minutes. In some cases, a non-natural RT provided herein retains at least 75% specific activity after incubation at a temperature of at least about 60° C. for at least 30 minutes. In some cases, a non-natural RT provided herein retains at least 75% specific activity after incubation at a temperature of at least about 60° C. for at least 45 minutes. In some cases, a non-natural RT provided herein retains at least 75% specific activity after incubation at a temperature of at least about 60° C. for at least 1 hour. In some cases, a non-natural RT provided herein retains at least 75% specific activity after incubation at a temperature of at least about 60° C. for at least 2 hours. In some cases, a non-natural RT provided herein retains at least 75% specific activity after incubation at a temperature of at least about 60° C. for at least 3 hours. In some cases, a non-natural RT provided herein retains at least 75% specific activity after incubation at a temperature of at least about 60° C. for at least 6 hours.

In some cases, a non-natural RT provided herein may retain at least about 5%, 10%, 15%, 20%, 25%, 30%, 35%, 40%, 45%, 50%, 55%, 60%, 65%, 70%, 75%, 80%, 85%, 90%, 95% or greater than 95% specific activity when conducting a reverse transcription reaction at an elevated temperature (e.g., temperatures of at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., at least about 40° C., at least about 45° C., at least about 50° C., at least about 55° C., at least about 60° C., at least about 65° C., at least about 70° C., at least about 75° C., at least about 80° C., at least about 85° C., or at least about 90° C.). In some cases, a non-natural RT provided herein may retain at least about 5%, 10%, 15%, 20%, 25%, 30%, 35%, 40%, 45%, 50%, 55%, 60%, 65%, 70%, 75%, 80%, 85%, 90%, 95% or greater than 95% specific activity when conducting a reverse transcription reaction at an elevated temperature for at least about 1 minute, at least about 2 minutes, at least about 3 minutes, at least about 4 minutes, at least about 5 minutes, at least about 10 minutes, at least about 15 minutes, at least about 20 minutes, at least about 25 minutes, at least about 30 minutes, at least about 45 minutes, at least about an hour, at least about a day, at least about 2 days, at least about 3 days, at least about 4 days, at least about 5 days, at least about 6 days, or at least about 1 week. For example, such timeframe may be between 30 minutes and 1 day, 30 minutes and 75 minutes, etc. In some cases, a non-natural RT provided herein retains at least 25% specific activity when conducting reverse transcription at a temperature of at least about 37° C. for at least 15 minutes. In some cases, a non-natural RT provided herein retains at least 25% specific activity when conducting reverse transcription at a temperature of at least about 37° C. for at least 30 minutes. In some cases, a non-natural RT provided herein retains at least 25% specific activity when conducting reverse transcription at a temperature of at least about 37° C. for at least 45 minutes. In some cases, a non-natural RT provided herein retains at least 25% specific activity when conducting reverse transcription at a temperature of at least about 37° C. for at least 1 hour. In some cases, a non-natural RT provided herein retains at least 25% specific activity when conducting reverse transcription at a temperature of at least about 37° C. for at least 2 hours. In some cases, a non-natural RT provided herein retains at least 25% specific activity when conducting reverse transcription at a temperature of at least about 37° C. for at least 3 hours. In some cases, a non-natural RT provided herein retains at least 25% specific activity when conducting reverse transcription at a temperature of at least about 37° C. for at least 6 hours. In some cases, a non-natural RT provided herein retains at least 50% specific activity when conducting reverse transcription at a temperature of at least about 37° C. for at least 15 minutes. In some cases, a non-natural RT provided herein retains at least 50% specific activity when conducting reverse transcription at a temperature of at least about 37° C. for at least 30 minutes. In some cases, a non-natural RT provided herein retains at least 50% specific activity when conducting reverse transcription at a temperature of at least about 37° C. for at least 45 minutes. In some cases, a non-natural RT provided herein retains at least 50% specific activity when conducting reverse transcription at a temperature of at least about 37° C. for at least 1 hour. In some cases, a non-natural RT provided herein retains at least 50% specific activity when conducting reverse transcription at a temperature of at least about 37° C. for at least 2 hours. In some cases, a non-natural RT provided herein retains at least 50% specific activity when conducting reverse transcription at a temperature of at least about 37° C. for at least 3 hours. In some cases, a non-natural RT provided herein retains at least 50% specific activity when conducting reverse transcription at a temperature of at least about 37° C. for at least 6 hours. In some cases, a non-natural RT provided herein retains at least 75% specific activity when conducting reverse transcription at a temperature of at least about 37° C. for at least 15 minutes. In some cases, a non-natural RT provided herein retains at least 75% specific activity when conducting reverse transcription at a temperature of at least about 37° C. for at least 30 minutes. In some cases, a non-natural RT provided herein retains at least 75% specific activity when conducting reverse transcription at a temperature of at least about 37° C. for at least 45 minutes. In some cases, a non-natural RT provided herein retains at least 75% specific activity when conducting reverse transcription at a temperature of at least about 37° C. for at least 1 hour. In some cases, a non-natural RT provided herein retains at least 75% specific activity when conducting reverse transcription at a temperature of at least about 37° C. for at least 2 hours. In some cases, a non-natural RT provided herein retains at least 75% specific activity when conducting reverse transcription at a temperature of at least about 37° C. for at least 3 hours. In some cases, a non-natural RT provided herein retains at least 75% specific activity when conducting reverse transcription at a temperature of at least about 37° C. for at least 6 hours. In some cases, a non-natural RT provided herein has at least 100% or greater specific activity when conducting reverse transcription at a temperature of at least about 37° C. for at least 15 minutes. In some cases, a non-natural RT provided herein has at least 100% or greater specific activity when conducting reverse transcription at a temperature of at least about 37° C. for at least 30 minutes. In some cases, a non-natural RT provided herein has at least 100% or greater specific activity when conducting reverse transcription at a temperature of at least about 37° C. for at least 45 minutes. In some cases, a non-natural RT provided herein has at least 100% or greater specific activity when conducting reverse transcription at a temperature of at least about 37° C. for at least 1 hour. In some cases, a non-natural RT provided herein has at least 100% or greater specific activity when conducting reverse transcription at a temperature of at least about 37° C. for at least 2 hours. In some cases, a non-natural RT provided herein has at least 100% or greater specific activity when conducting reverse transcription at a temperature of at least about 37° C. for at least 3 hours. In some cases, a non-natural RT provided herein retains has at least 100% or greater specific activity when conducting reverse transcription at a temperature of at least about 37° C. for at least 6 hours. In some cases, a non-natural RT provided herein has at least 150% or greater specific activity when conducting reverse transcription at a temperature of at least about 37° C. for at least 15 minutes. In some cases, a non-natural RT provided herein has at least 150% or greater specific activity when conducting reverse transcription at a temperature of at least about 37° C. for at least 30 minutes. In some cases, a non-natural RT provided herein has at least 150% or greater specific activity when conducting reverse transcription at a temperature of at least about 37° C. for at least 45 minutes. In some cases, a non-natural RT provided herein has at least 150% or greater specific activity when conducting reverse transcription at a temperature of at least about 37° C. for at least 1 hour. In some cases, a non-natural RT provided herein has at least 150% or greater specific activity when conducting reverse transcription at a temperature of at least about 37° C. for at least 2 hours. In some cases, a non-natural RT provided herein has at least 150% or greater specific activity when conducting reverse transcription at a temperature of at least about 37° C. for at least 3 hours. In some cases, a non-natural RT provided herein retains has at least 150% or greater specific activity when conducting reverse transcription at a temperature of at least about 37° C. for at least 6 hours.

A non-natural RT provided herein may generate a high yield of cDNA when conducting reverse transcription at elevated temperatures (e.g., temperatures of at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., at least about 40° C., at least about 45° C., at least about 50° C., at least about 55° C., at least about 60° C., at least about 65° C., at least about 70° C., at least about 75° C., at least about 80° C., at least about 85° C., or at least about 90° C.). In some cases, such temperature is between 65° C. and 75° C. For example, at elevated temperatures, a non-natural RT of the disclosure may generate a quantity of cDNA transcript (e.g, partial and/or full-length) that is at least about 25%, 30%, 35%, 40%, 45%, 50%, 55%, 60%, 65%, 70%, 75%, 80%, 85%, 90%, 95%, 100%, 150%, 200%, 250%, or at least about 300% of the amount of cDNA transcript (e.g., partial and/or full-length) produced by the same non-natural RT at a lower temperature (e.g., less than 37° C., less than 35° C.). In some cases, a non-natural RT of the disclosure may produce about the same amount of cDNA transcript at a lower temperature compared to the amount of cDNA product made by the same non-natural RT at the higher temperature. In some embodiments, a non-natural RT of the disclosure generates at least about 25% of the amount cDNA transcript (e.g., partial and/or full-length) produced by the same non-natural RT at a lower temperature (e.g., less than 37° C.). In some embodiments, a non-natural RT of the disclosure generates at least about 50% of the amount cDNA transcript (e.g., partial and/or full-length) produced by the same non-natural RT at a lower temperature (e.g., less than 37° C.). In some embodiments, a non-natural RT of the disclosure generates at least about 75% of the amount cDNA transcript (e.g., partial and/or full-length) produced by the same non-natural RT at a lower temperature (e.g., less than 37° C.). In some embodiments, a non-natural RT of the disclosure generates about 100% of the amount cDNA transcript (e.g., partial and/or full-length) produced by the same non-natural RT at a lower temperature (e.g., less than 37° C.).

The quantity of cDNA produced by a non-natural RT (e.g., chimeric) at an elevated temperature (e.g., temperatures of at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., at least about 40° C., at least about 45° C., at least about 50° C., at least about 55° C., at least about 60° C., at least about 65° C., at least about 70° C., at least about 75° C., at least about 80° C., at least about 85° C., or at least about 90° C.) may also be superior to that produced by a natural RT during a similar timeframe. In some cases, the amount of partial and/or full length cDNA transcript produced by a non-natural RT of the present disclosure at elevated reaction temperature (e.g., temperatures of at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., at least about 40° C., at least about 45° C., at least about 50° C., at least about 55° C., at least about 60° C., at least about 65° C., at least about 70° C., at least about 75° C., at least about 80° C., at least about 85° C., or at least about 90° C.) may be at least about 1.5-fold, 2-fold, 5-fold, 10-fold, 50-fold, 100-fold, 150-fold, 200-fold, 250-fold, 300-fold, 350-fold, 400-fold, 450-fold, 500-fold or greater than the amount of full-length product synthesized by a natural RT under similar conditions. In some cases, the amount of cDNA transcript (e.g., partial and/or full length) produced by a non-natural RT of the present disclosure at a reaction temperature of at least about 37° C. is at least about 50-fold greater than the amount of full-length product synthesized by a natural RT under similar conditions. In some cases, the amount of cDNA transcript (e.g., partial and/or full length) produced by a non-natural RT of the present disclosure at a reaction temperature of at least about 37° C. is at least about 100-fold greater than the amount of full-length product synthesized by a natural RT under similar conditions. In some cases, the amount of cDNA transcript (e.g., partial and/or full length) produced by a non-natural RT of the present disclosure at a reaction temperature of at least about 37° C. is at least about 150-fold greater than the amount of full-length product synthesized by a natural RT under similar conditions. In some cases, the amount of cDNA transcript (e.g., partial and/or full length) produced by a non-natural RT of the present disclosure at a reaction temperature of at least about 42° C. is at least about 50-fold greater than the amount of full-length product synthesized by a natural RT under similar conditions. In some cases, the amount of cDNA transcript (e.g., partial and/or full length) produced by a non-natural RT of the present disclosure at a reaction temperature of at least about 42° C. is at least about 100-fold greater than the amount of full-length product synthesized by a natural RT under similar conditions. In some cases, the amount of cDNA transcript (e.g., partial and/or full length) produced by a non-natural RT of the present disclosure at a reaction temperature of at least about 42° C. is at least about 150-fold greater than the amount of full-length product synthesized by a natural RT under similar conditions.

Non-natural (e.g., chimeric) RTs of the present disclosure may exhibit increased thermostability in the presence or absence of an RNA template. In some instances, chimeric RTs of the disclosure may show an increased thermostability in both the presence and absence of an RNA template. The increase in thermostability may be measured by comparing suitable parameters of the chimeric RTs to those of a corresponding non-chimeric RTs.

In some cases, thermostability can be determined by pre-incubating the non-natural or chimeric RT at elevated temperatures (e.g., temperatures of at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., at least about 40° C., at least about 45° C., at least about 50° C., at least about 55° C., at least about 60° C., at least about 65° C., at least about 70° C., at least about 75° C., at least about 80° C., at least about 85° C., or at least about 90° C.) and subsequently determining the ability of the non-natural RTs to produce partial and/or full length cDNA product at a certain reaction temperature, for example a reaction temperature which is lower than the pre-incubation temperature. Pre-incubation steps may be used, for example, during reverse transcription to reduce the presence of RNA secondary structure since reduction of secondary structure may increase the cDNA yield or reduce errors. The duration of a pre-incubation step can be any reasonable period of time (e.g., at least about 5 minutes, at least about 10 minutes, at least about 15 minutes, at least about 20 minutes, at least about 25 minutes, at least about 30 minutes, at least about 45 minutes, at least about an hour or longer than an hour). Following the pre-incubation and reverse transcription, the cDNA can be amplified, for example by PCR amplification and then analyzed.

Thermal stability of proteins can be investigated using various other methods including but not limited to fluorescence analysis, biochemical assays, circular dichroism, hydrogen exchange-mass spectroscopy, protein crystallization, and/or differential scanning calorimetry. Thermal stability may also be determined, for example in the case of enzymatic proteins, by assaying for enzymatic activity at varying temperatures.

A non-natural RT provided herein may exhibit particularly high specific activity. As used herein, the term specific activity is generally described in units/mg (U/mg) or units/μg (U/μg) wherein one unit (U) refers to the amount of enzyme that incorporates 1 nmol of dTTP in a RNA-directed DNA polymerization reaction using a poly(rA) as a template and an oligo(dT) as a primer in about 10 minutes at a reaction temperature of about 37° C. Various methods for measuring RT activity are available and include for example, radioactive nucleotide or fluorophore-labeled nucleotide incorporation assays.

In some embodiments, a non-natural RT provided herein may have a RNA-directed DNA polymerase specific activity of at least about 150 U/μg, at least about 200 U/μg, at least about 250 U/μg, at least about 300 U/μg, at least about 350 U/μg, at least about 400 U/μg, at least about 450 U/μg, at least about 500 U/μg, at least about 550 U/μg, at least about 600 U/μg, at least about 650 U/μg, at least about 700 U/μg, at least about 750 U/μg, at least about 800 U/μg, at least about 850 U/μg, at least about 900 U/μg, at least about 950 U/μg, at least about 1,000 U/μg, at least about 1,100 U/μg, at least about 1,200 U/μg, at least about 1,300 U/μg, at least about 1,400 U/μg, at least about 1,500 U/μg, at least about 1,600 U/μg, at least about 1,700 U/μg, at least about 1,800 U/μg, at least about 1,900 U/μg, at least about 2,000 U/μg, at least about 2,500 U/μg, at least about 3,000 U/μg, at least about 3,500 U/μg, at least about 4,000 U/μg, at least about 5,000 U/μg, at least about 6,000 U/μg, at least about 7,000 U/μg, at least about 8,000 U/μg, at least about 9,000 U/μg, at least about 10,000 U/μg, at least about 11,000 U/μg, at least about 12,000 U/μg, at least about 13,000 U/μg, at least about 14,000 U/μg, at least about 15,000 U/μg, at least about 16,000 U/μg, at least about 17,000 U/μg, at least about 18,000 U/μg, at least about 19,000 U/μg, at least about 20,000 U/μg, at least about 21,000 U/μg, at least about 22,000 U/μg, at least about 23,000 U/μg, at least about 24,000 U/μg, at least about 25,000 U/μg, at least about 26,000 U/μg, at least about 27,000 U/μg, at least about 28,000 U/μg, at least about 29,000 U/μg, at least about 30,000 U/μg, at least about 31,000 U/μg, at least about 32,000 U/μg, at least about 33,000 U/μg, at least about 34,000 U/μg, at least about 35,000 U/μg, at least about 36,000 U/μg, at least about 37,000 U/μg, at least about 38,000 U/μg, at least about 39,000 U/μg, at least about 40,000 U/μg, at least about 41,000 U/μg, at least about 42,000 U/μg, at least about 43,000 U/μg, at least about 44,000 U/μg, at least about 45,000 U/μg, at least about 50,000 U/μg, at least about 100,000 U/μg, at least about 150,000 U/μg, at least about 200,000 U/μg, or at least about 250,000 U/μg. In some instances, the non-natural RT exhibits a specific activity of at least about 250 U/μg. In some examples, the non-natural RT exhibits a specific activity of at least about 450 U/μg. In some instances, the non-natural RT exhibits a specific activity of at least about 800 U/μg. In some instances, the non-natural RT exhibits a specific activity of at least about 1,000 U/μg. In some instances, the non-natural RT exhibits a specific activity of at least about 5,000 U/μg. In some instances, the non-natural RT exhibits a specific activity of at least about 10,000 U/μg. In some instances, the non-natural RT exhibits a specific activity of less than about 10,000 U/μg. In some instances, the non-natural RT exhibits a specific activity of at least about 15,000 U/μg. In some instances, the non-natural RT exhibits a specific activity of at least about 20,000 U/μg. In some instances, the non-natural RT exhibits a specific activity of less than about 20,000 U/μg. In some instances, the non-natural RT exhibits a specific activity of at least about 25,000 U/μg. In some instances, the non-natural RT exhibits a specific activity of at least about 30,000 U/μg. In some instances, the non-natural RT exhibits a specific activity of at least about 35,000 U/μg. In some instances, the non-natural RT exhibits a specific activity of at least about 40,000 U/μg. In some instances, the non-natural RT exhibits a specific activity of at least about 45,000 U/μg. In some instances, the non-natural RT exhibits a specific activity of at least about 10,000 U/μg to about 45,000 U/μg.

A non-natural RT disclosed herein may exhibit a high specific activity in comparison to a wild-type RT, particularly a natural or wild-type form of the RT. In some cases, a non-natural RT disclosed herein may have specific activity that is at least about 5% greater than its natural (e.g., wild-type or natural) form, for example, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 150%, at least about 200%, at least about 250%, at least about 300%, at least about 350%, at least about 400%, or at least about 450% greater than its natural form. In some cases, a non-natural or chimeric reverse transcriptase may have specific activity that is at least about 0.1-fold, 0.2-fold, 0.3-fold, 0.4-fold, 0.5-fold, 1-fold, 2-fold, 3-fold, 4-fold, 5-fold, 10-fold, 20-fold, 30-fold, 40-fold, 50-fold, 60-fold, 70-fold, 80-fold, 90-fold, 100-fold, 110-fold, 120-fold, 130-fold, 140-fold, 150-fold, 160-fold, 170-fold, 180-fold, 190-fold, 200-fold, 250-fold, 300-fold, 350-fold, 400-fold, 450-fold, or 500-fold the specific activity of its wild-type form. A non-natural RT that has greater reverse transcriptase activity than a natural RT may be able to better generate cDNA product from RNA template that is of low quality and/or low quantity.

A non-natural RT provided herein may exhibit high processivity as it may promote the generation of cDNA (single-stranded or double-stranded) with a particularly long length. For example, a non-natural or chimeric RT provided herein may be able to synthesize partial and/or full length cDNA product at least about 500 bp (e.g., at least about 1 kb, at least about 2 kb, at least about 3 kb, at least about 4 kb, at least about 5 kb, at least about 6 kb, at least about 7 kb, at least about 8 kb, at least about 9 kb, at least about 10 kb or longer than 10 kb) in length. In some instances, a non-natural or chimeric RT provided herein may be able to synthesize cDNA product and/or full length cDNA product between about 500 bp and 15 kb in length (e.g., between about 750 bp and 12.5 kb in length, between about 1 kb and 10 kb in length, between about 2 kb and 9 kb in length, between about 3 kb and 8 kb in length, between about 4 kb and 7 kb in length, or between about 5 kb and 6 kb in length).

In some cases, a non-natural RT provided herein may exhibit high efficiency, high accuracy, high yield, high specificity and/or high fidelity, particularly when compared with a natural RT. Efficiency, for example, may be determined, by quantifying the cDNA yield from reverse transcription for a given amount of template RNA. Efficiency can be determined relative to a reference, for example the efficiency of a non-natural RT can be determined relative to the efficiency of a natural RT. In some cases, efficiency may refer to the proportion of full-length product to total product (e.g., full-length and partial product). In some cases, efficiency may refer to the ability of a RT to reverse transcribe low quality and/or low quantities of RNA template. Efficiency may be determined by quantifying cDNA yield during reverse transcription, for example using quantitative reverse transcription PCR or RT-qPCR, and/or at the completion of reverse transcription, for example using an agarose gel for quantification or other DNA detection method, such as with a spectrophotometer.

In some examples, the efficiency of the RTs described herein may be increased relative to non-chimeric RTs (e.g., natural, wild-type RTs), by at least 5%, 10%, 15%, 20%, 25%, 30%, 35%, 40%, 45%, 50%, 55%, 60%, 65%, 70%, 75%, 80%, 85%, 90%, 95% or greater than 95%. In some cases, a non-natural or chimeric reverse transcriptase may have an efficiency that is at least about 0.1-fold, 0.2-fold, 0.3-fold, 0.4-fold, 0.5-fold, 1-fold, 2-fold, 3-fold, 4-fold, 5-fold, 10-fold or greater than 10-fold the efficiency of its wild-type or natural form. In such examples in particular, the non-natural RT may exhibit a greater yield of cDNA product compared to, for example, a natural RT.

### Peptide Tag

In some embodiments, at least one peptide tag is linked to non-natural (e.g., chimeric) RTs and other proteins (e.g., enzymes) of the disclosure. The at least one peptide tag may function as stability enhancing tag. The non-natural RTs herein may be linked to at least two (e.g., at least three, four, five or more) peptide tags which function as stability enhancing tags. Stability enhancing peptide tags may enhance the stability of proteins (e.g., thermostable enzymes, non-thermostable enzymes) following short-term or long-term exposure to a temperature between about −20° C. and about +65° C., between about −20° C. and about +60° C., between about −20° C. and about +55° C., between about −20° C. and about +50° C., between about −20° C. and about +45° C., between about −20° C. and about +40° C., between about −20° C. and about +35° C., between about −20° C. and about +30° C., between about −20° C. and about +25° C., between about −20° C. and about +20° C., between about −20° C. and about +15° C., between about −20° C. and about +10° C., between about −20° C. and about +5° C., between about −20° C. and about 0° C., between about −20° C. and about +65° C., between about −15° C. and about +65° C., between about −10° C. and about +65° C., between about −5° C. and about +65° C., between about 0° C. and about +65° C., between about 5° C. and about +65° C., between about 10° C. and about +65° C., between about 15° C. and about +65° C., between about 20° C. and about +65° C., between about 25° C. and about +65° C., between about 30° C. and about +65° C., between about 35° C. and about +65° C., between about 40° C. and about +65° C., between about 45° C. and about +65° C., between about 50° C. and about +65° C., between about 55° C. and about +65° C., or between about 60° C. and about +65° C. In some cases, the stability enhancing peptide tags can enhance the stability of proteins following exposure to various temperatures above room temperature (e.g., temperatures of at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., at least about 40° C., at least about 45° C., at least about 50° C., at least about 55° C., at least about 60° C., at least about 65° C., at least about 70° C., at least about 75° C., at least about 80° C., at least about 85° C., or at least about 90° C.). The peptide tags may aid the retention of protein structure, stability, enzymatic activity, binding activity, any other property, or any combination thereof. In some embodiments, the chimeric RTs linked to stability enhancing peptide tags may demonstrate enhanced stability or enzymatic activity when compared to a similar protein that does not have the tag, especially after short-term or long-term exposure to a certain temperature (e.g., temperatures of room temperature or above).

In some cases, a stability enhancing peptide tag comprises a motif comprising at least one to six histidine residues. In some cases, a stability enhancing peptide comprises a protease cleavage site comprising the amino acid sequence DDDDK (SEQ ID NO: 19). A stability enhancing peptide can comprise the conserved sequence HHHIHHHIPWDYKDDDDKPRWNS (SEQ ID NO: 20), which includes six histidine residues and a protease cleavage site. A stability enhancing peptide tag may be of any suitable length, e.g., at least 20 amino acids, at least 25 amino acids, at least 30 amino acids, at least 35 amino acids, at least 40 amino acids, at least 45 amino acids, or at least 50 amino acids in length. In some cases, a stability enhancing peptide tag may comprise the sequence MIDLQRPQAATMDSRHHHHHHPWDYKDDDDKPRWNS (SEQ ID NO: 18). A stability enhancing tag may comprise a sequence that is at least 75%, 80%, 85%, 90%, 95%, 96%, 97%, 98%, or 99% identical to SEQ ID NO: 18 and comprises the sequence of SEQ ID NO: 20.

In some embodiments, a non-natural (e.g., chimeric) RT comprises a ribonuclease domain from T. thermophilus or a variant thereof (e.g., a variant having reduced ribonuclease activity) linked to a polymerase domain from M-MLV RT or a variant thereof which is further linked to a stability enhancing peptide tag (SEQ ID NO: 14, the peptide tag sequence is indicated by the underlined sequence). In some embodiments, a chimeric RT comprises a ribonuclease domain from T. litoralis (e.g., RNase H II) or a variant thereof (e.g., a variant having reduced ribonuclease activity) linked to a polymerase domain from M-MLV RT or a variant thereof which is further linked to a stability enhancing peptide tag (SEQ ID NO: 15, the peptide tag sequence is indicated by the underlined sequence). In some embodiments, a chimeric RT comprises a ribonuclease domain from T. gammatolerans or a variant thereof (e.g., a variant having reduced ribonuclease activity) linked to a polymerase domain from M-MLV RT or a variant thereof which is further linked to a stability enhancing peptide tag.

In some embodiments, a chimeric reverse transcriptase comprises a ribonuclease domain from T. thermophilus or a variant thereof (e.g., a variant having reduced ribonuclease activity) linked to a polymerase domain from HIV-1 RT or a variant thereof which is further linked to a stability enhancing peptide tag (SEQ ID NO: 16, the peptide tag sequence is indicated by the underlined sequence). In some embodiments, a chimeric RT comprises a ribonuclease domain from T. litoralis (e.g., RNase H II) or a variant thereof (e.g., a variant having reduced ribonuclease activity) linked to a polymerase domain from HIV-1 RT or a variant thereof which is further linked to a stability enhancing peptide tag (SEQ ID NO: 17, the peptide tag sequence is indicated by the underlined sequence). In some embodiments, a chimeric RT comprises a ribonuclease domain from T. gammatolerans or a variant thereof (e.g., a variant having reduced ribonuclease activity) linked to a polymerase domain from HIV-1 RT or a variant thereof which is further linked to a stability enhancing peptide tag.

In some cases, a chimeric reverse transcriptase may comprise an amino acid sequence of any of SEQ ID NOs: 14-17. In some cases, a chimeric reverse transcriptase may comprise an amino acid sequence at least 75%, at least 80%, at least 85%, at least 90%, at least 91%, at least 92%, at least 93%, at least 94%, at least 95%, at least 96%, at least 97%, at least 98%, or at least 99% identical to of any of SEQ ID NOs: 14-17.

TABLE 3
Amino acid sequence of chimeric RTs
including stability enhancing peptide tag

| SEQ | | |
|---|---|---|
| ID NO: | Description | Amino Acid Sequence |
| 13 | Tag-wt M- | MIDLQRPQAATMDSRHHHHHHPWDYKDDDDKPRWNSLN |
| | MLV | IEDEHRLHETSKEPDVSLGSTWLSDFPQAWAETGGMGLAV |
| | | RQAPLIIPLKATSTPVSIKQYPMSQEARLGIKPHIQRLLDQGI |
| | | LVPCQSPWNTPLLPVKKPGTNDYRPVQDLREVNKRVEDIH |
| | | PTVPNPYNLLSGLPPSHQWYTVLDLKDAFFCLRLHPTSQPL |
| | | FAFEWRDPEMGISGQLTWTRLPQGFKNSPTLFDEALHRDL |
| | | ADFRIQHPDLILLQYVDDLLLAATSELDCQQGTRALLQTLG |
| | | NLGYRASAKKAQICQKQVKYLGYLLKEGQRWLTEARKET |
| | | VMGQPTPKTPRQLREFLGTAGFCRLWIPGFAEMAAPLYPL |
| | | TKTGTLFNWGPDQQKAYQEIKQALLTAPALGLPDLTKPFE |
| | | LFVDEKQGYAKGVLTQKLGPWRRPVAYLSKKLDPVAAG |
| | | WPPCLRMVAAIAVLTKDAGKLTMGQPLVILAPHAVEALV |
| | | KQPPDRWLSNARMTHYQALLLDTDRVQFGPVVALNPATL |
| | | LPLPEEGLQHNCLDILAEAHGTRPDLTDQPLPDADHTWYT |
| | | DGSSLLQEGQRKAGAAVTTETEVIWAKALPAGTSAQRAEL |
| | | IALTQALKMAEGKKLNVYTDSRYAFATAHIHGEIYRRRGL |
| | | LTSEGKEIKNKDEILALLKALFLPKRLSIIHCPGHQKGHSAE |
| | | ARGNRMADQAARKAAITETPDTSTLLI |
| 14 | Tag-M-MLV | MIDLQRPQAATMDSRHHHHHHPWDYKDDDDKPRWNSLN |
| | pol domain-Tth | IEDEHRLHETSKEPDVSLGSTWLSDFPQAWAETGGMGLAV |
| | RNase H⁻ | RQAPLIIPLKATSTPVSIKQYPMSQEARLGIKPHIQRLLDQGI |
| | | LVPCQSPWNTPLLPVKKPGTNDYRPVQDLREVNKRVEDIH |
| | | PTVPNPYNLLSGLPPSHQWYTVLDLKDAFFCLRLHPTSQPL |
| | | FAFEWRDPEMGISGQLTWTRLPQGFKNSPTLFDEALHRDL |
| | | ADFRIQHPDLILLQYVDDLLLAATSELDCQQGTRALLQTLG |
| | | NLGYRASAKKAQICQKQVKYLGYLLKEGQRWLTEARKET |
| | | VMGQPTPKTPRQLREFLGTAGFCRLWIPGFAEMAAPLYPL |
| | | TKTGTLFNWGPDQQKAYQEIKQALLTAPALGLPDLTKPFE |
| | | LFVDEKQGYAKGVLTQKLGPWRRPVAYLSKKLDPVAAG |
| | | WPPCLRMVAAIAVLTKDAGKLTMGQPLVILAPHAVEALV |
| | | KQPPDRWLSNARMTHYQALLLDTDRVQFGPVVALNPATL |
| | | LPLPEEGLQHNCLDILAEAHGTRPDLTDQPLPDNPSPRKRV |
| | | ALFTNGACLGNPGPGGWAALLRFHAHEKLLSGGEACT |
| | | TNNRMELKAAIEGLKALKEPCEVDLYTDSHYLKKAFTE |
| | | GWLEGWRKRGWRTAEGKPVKNRDLWEALLLAMAPH |
| | | RVRFHFVKGHTGHPENERVDREARRQAQSQAKTPCPP |
| | | RAPTLFHEEA |
| 15 | Tag-M-MLV | MIDLQRPQAATMDSRHHHHHHPWDYKDDDDKPRWNSLN |
| | pol domain-Tli | IEDEHRLHETSKEPDVSLGSTWLSDFPQAWAETGGMGLAV |
| | Rnase H II⁻ | RQAPLIIPLKATSTPVSIKQYPMSQEARLGIKPHIQRLLDQGI |
| | | LVPCQSPWNTPLLPVKKPGTNDYRPVQDLREVNKRVEDIH |
| | | PTVPNPYNLLSGLPPSHQWYTVLDLKDAFFCLRLHPTSQPL |
| | | FAFEWRDPEMGISGQLTWTRLPQGFKNSPTLFDEALHRDL |
| | | ADFRIQHPDLILLQYVDDLLLAATSELDCQQGTRALLQTLG |
| | | NLGYRASAKKAQICQKQVKYLGYLLKEGQRWLTEARKET |
| | | VMGQPTPKTPRQLREFLGTAGFCRLWIPGFAEMAAPLYPL |
| | | TKTGTLFNWGPDQQKAYQEIKQALLTAPALGLPDLTKPFE |
| | | LFVDEKQGYAKGVLTQKLGPWRRPVAYLSKKLDPVAAG |
| | | WPPCLRMVAAIAVLTKDAGKLTMGQPLVILAPHAVEALV |
| | | KQPPDRWLSNARMTHYQALLLDTDRVQFGPVVALNPATL |
| | | LPLPEEGLQHNCLDILAEAHGTRPDLTDQPLPDKLGGINQ |
| | | AGRGPVIGPLVIAAVVVDESRMQELEALGVKDSKKLTP |
| | | KRREELFEEIVQIVDDHVIIQLSPEEIDGRDGTMNELEIE |
| | | NFAKALNSLKVKPDVLYIDAADVKEKRFGDIIGERLSFS |
| | | PKIIAEHKADSKYIPVAAASILAKVTRDRAIEKLKELYGE |
| | | IGSGYPSDPNTRRFLEEYYKAHGEFPPIVRKSWKTLRKI |
| | | EEKLKAKKTQPTILDFLKKP |
| 16 | Tag-HIV-1 pol | MIDLQRPQAATMDSRHHHHHHPWDYKDDDDKPRWNSNF |
| | domain-Tth | PISPIETVPVKLKPGMDGPKVKQWPLTEEKIKALVEICTEM |
| | Rnase H⁻ | EKEGKISKIGPENPYNTPVFAIKKKDSTKWRKLVDFRELNK |
| | | RTQDFWEVQLGIPHPAGLKKKKSVTVLDVGDAYFSVPLDE |
| | | DFRKYTAFTIPSINNETPGIRYQYNVLPQGWKGSPAIFQSSM |
| | | TKILEPFRKQNPDIVIYQYMDDLYVGSDLEIGQHRTKIEELR |
| | | QHLLRWGLTTPDKKHQKEPPFLWMGYELHPDKWTVQPIV |
| | | LPEKDSWTVNDIQKLVGKLNWASQIYPGIKVRQLCKLLRG |
| | | TKALTEVIPLTEEAELELAENREILKEPVHGVYYDPSKDLIA |
| | | EIQKQGQGQWTYQIYQEPFKNLKTGKYARMRGAHTNDVK |
| | | QLTEAVQKITTESIVIWGKTPKFKLPIQKETWETWWTEYW |
| | | QATWIPEWEFVNTPPLVKLWYQLEKEPIVNPSPRKRVALF |
| | | TNGACLGNPGPGGWAALLRFHAHEKLLSGGEACTTNN |
| | | RMELKAAIEGLKALKEPCEVDLYTDSHYLKKAFTEGW |
| | | LEGWRKRGWRTAEGKPVKNRDLWEALLLAMAPHRV |
| | | RFHFVKGHTGHPENERVDREARRQAQSQAKTPCPPRA |
| | | PTLFHEEA |
| 17 | Tag-HIV-1 pol | MIDLQRPQAATMDSRHHHHHHPWDYKDDDDKPRWNSNF |
| | domain-Tli | PISPIETVPVKLKPGMDGPKVKQWPLTEEKIKALVEICTEM |
| | Rnase H II⁻ | EKEGKISKIGPENPYNTPVFAIKKKDSTKWRKLVDFRELNK |
| | | RTQDFWEVQLGIPHPAGLKKKKSVTVLDVGDAYFSVPLDE |
| | | DFRKYTAFTIPSINNETPGIRYQYNVLPQGWKGSPAIFQSSM |
| | | TKILEPFRKQNPDIVIYQYMDDLYVGSDLEIGQHRTKIEELR |
| | | QHLLRWGLTTPDKKHQKEPPFLWMGYELHPDKWTVQPIV |
| | | LPEKDSWTVNDIQKLVGKLNWASQIYPGIKVRQLCKLLRG |
| | | TKALTEVIPLTEEAELELAENREILKEPVHGVYYDPSKDLIA |
| | | EIQKQGQGQWTYQIYQEPFKNLKTGKYARMRGAHTNDVK |
| | | QLTEAVQKITTESIVIWGKTPKFKLPIQKETWETWWTEYW |
| | | QATWIPEWEFVNTPPLVKLWYQLEKEPIVKLGGINQAGR |
| | | GPVIGPLVIAAVVVDESRMQELEALGVKDSKKLTPKRR |
| | | EELFEEIVQIVDDHVIIQLSPEEIDGRDGTMNELEIENFA |
| | | KALNSLKVKPDVLYIDAADVKEKRFGDIIGERLSFSPKII |
| | | AEHKADSKYIPVAAASILAKVTRDRAIEKLKELYGEIGS |
| | | GYPSDPNTRRFLEEYYKAHGEFPPIVRKSWKTLRKIEE |
| | | KLKAKKTQPTILDFLKKP |
| 18 | Tag | MIDLQRPQAATMDSRHHHHHHPWDYKDDDDKPRWNS |
| 19 | Protease | DDDDK |
| | cleavage site | |
| 20 | Peptide tag | HHHHHHPWDYKDDDDKPRWNS |

In some embodiments, the present disclosure provides kits comprising one or more non-natural (e.g., chimeric) RTs disclosed herein. The chimeric RTs may be directly formulated into compositions and provided in kits for conducting assays involving reverse transcription. A kit comprising non-natural RTs disclosed herein may include the chimeric RTs formulated as a solution (for example an aqueous solution, a glycerol solution, etc) or as a lyophilized powder which is reconstituted prior to use or according to other instructions provided with the kit. A kit may include a first container (e.g., tube, vial, ampule) that contains one or more non-natural (e.g., chimeric) RTs disclosed herein and at least a second container containing one or more other components (e.g., polymerase, DNA polymerase, DNA-dependent DNA polymerase, dNTPs, buffer for nucleic acid synthesis, reagent, dye). The individual components of the kit described herein can be present in separate containers, or some components may be present as a mixture in the same container. In preferred embodiments the kits provided herein contain a DNA-dependent DNA polymerase, which may, in some cases, be present in a separate container from the chimeric RT. In some cases, the DNA-dependent DNA polymerase is mixed with the chimeric RT in the same container. The DNA-dependent DNA polymerase may be present in solution or, for example, as a lyophilized powder.

Kits comprising non-natural, chimeric RTs for conducting assays involving reverse transcription may further comprise reaction media(s) or buffer(s) which can be optimized for reverse transcription or enzyme storage. Appropriate reaction media or buffers for kits comprising chimeric RTs may permit reverse transcription with a pre-incubation step and/or at elevated reaction temperatures, and optionally nucleic acid amplification according to the methods provided herein (e.g., in 1 step RT-PCR or 2 step RT-PCR reactions). The pH of the media or buffer may be from about pH 5 to about pH 11, from about pH 6 to about pH 10, from about pH 7 to about pH 9, or from about pH 7.5 to about pH 8.5. More acidic and alkaline buffers may also be used. The pH of the media or buffer can be adjusted, for example, to optimize the enzymatic activity of either the chimeric RT and/or a DNA polymerase, if one is provided. The media or buffer may comprise Tris(hydroxymethyl)aminomethane (Tris). In some embodiments, the media or buffer can include about 50-80 mM Tris (e.g., at a pH of about pH 8.3 to pH 9.0) and about 10-20 mM (NH4)2SO4 or about 30-50 mM KCl. Other media or buffers may also be used, e.g., so long as the components are non-inhibitory to the enzyme components of the kit or minimally inhibit the enzyme components of the kit or can be removed before using the enzyme.

A kit may comprise reaction medium or buffer having bivalent metal ions such as Mg2+ or Mn2+. The final concentration of free ions may be within a range of from about 0.01 mM to about 15 mM, or from about 1 mM to about 10 mM. In some embodiments, the reaction medium or buffer comprises MgCl2 (e.g, at least about 1 mM, 1.5 mM, 2 mM, 5 mM, 7.5 mM, 10 mM, 15 mM, 20 mM, 25 mM, 30 mM, 40 mM or 50 mM of MgCl2 or greater than 50 mM of MgCl2). In some embodiments, the reaction medium or buffer can also include additional salts such as KCl and/or NaCl. Additional salts may contribute to the total ionic strength of the medium. For example, the range of a salt such as KCl may be from about 0 mM to about 125 mM (e.g., from about 0 mM to about 100 mM, from about 0 mM to about 75 mM, or from about 0 mM to about 50 mM). For example, the range of a salt such as NaCl may be from about 0 mM to about 125 mM (e.g., from about 0 mM to about 100 mM, from about 0 mM to about 75 mM, or from about 0 mM to about 50 mM). The reaction medium or buffer can further include additives that could affect reverse transcription and/or amplification. Such additives include, but are not limited to, proteins such as bovine serum albumin (BSA), single strand binding proteins, and non-ionic detergents such as NP40 or Triton.

Kits comprising non-natural RTs provided herein may also comprise reagents (e.g., dithiothreitol or DTT), that are capable of maintaining enzyme activities. In some embodiments, the kits comprising non-natural RTs provided herein can include a linear polyacrylamide (LPA), which can increase the specificity and sensitivity of an enzyme.

In some embodiments, a kit comprises at least one primer for synthesizing cDNA from a template RNA. Primers for synthesizing cDNA from a template RNA may be provided in solution. A primer for synthesizing cDNA from a template RNA may not be limited to a specific sequence so long as the nucleotide sequence of the primer is complementary (e.g., partially or fully) to that of the template RNA and can anneal to the template RNA under desired reaction conditions. In some cases, the primer comprises an oligo(dT), or an oligonucleotide comprising ‘T’ bases. An oligo(dT) can hybridize to poly-A sequences such as, for example, those found on mRNA. In some cases, the primer comprises an oligonucleotide having a random sequence (e.g., a random primer). In some cases, the primer comprises an oligonucleotide having a gene-specific sequence. A kit may further include oligonucleotide primers for amplification processes other than reverse transcription, for example for cDNA amplification. Primers for amplification processes other than reverse transcription can be provided in solution. In some embodiments, oligonucleotides provided in kits disclosed herein may be modified, for example with labels such as fluorophores, fluorescent moieties, and dyes.

In some embodiments, a composition of a kit may also comprise nucleotides, such as deoxynucleotide triphosphates (dNTPs) and/or dideoxynucleotide triphosphates (ddNTPs) (e.g., at least about 1 mM, 1.5 mM, 2 mM, 5 mM, 7.5 mM, 10 mM, 15 mM, 20 mM, 25 mM, 30 mM, 40 mM or 50 mM dNTPs or ddNTPs). The dNTPs may be ultrapure dNTPs. The dNTPs may comprise dATP, dGTP, dCTP, dTTP, dUTP, or any combination thereof. The dNTPs provided in a kit may be modified dNTPs or dNTP analogs. In some cases, dNTPs and ddNTPs can include “labels” which can be used, either directly or in combination with other molecules such as reporter molecules, for the detection of cDNA and/or amplification products (e.g., in RT-qPCR or quantitative reverse transcription PCR). Labels can comprise molecular structures that, once attached to a nucleic acid, provide a distinct characteristic that is not inherent to the dNTPs, ddNTPs, cDNA or amplified polynucleotides. In some cases, nucleotide and nucleotide analogs, including ddNTPs, can comprise fluorophores or fluorescent moieties (e.g., dyes) as labels.

The kits disclosed herein may comprise a DNA-binding dye, such as dyes that bind double-stranded DNA and emit a signal (e.g., a fluorescent signal). Non-limiting examples of DNA binding dyes include EvaGreen™, described in U.S. Pat. No. 7,601,498; LC Green; SYTO9; Chromofy; BEBO; and SYBR Green. Such dyes may be useful for quantitative PCR (qPCR) applications. The kits may, in some cases, comprise a reference dye (e.g., ROX dye), or a quencher dye (e.g., TAMRA). In some embodiments, the above-mentioned reference dyes can be used in a variety of reactions, including reactions for real-time quantitative PCR including RT-qPCR.

A kit may further comprise an RNase inhibitor. An RNase inhibitor provided in a kit may inhibit RNases A, B, C, or any combination thereof. A RNase inhibitor provided in a kit may inhibit RNase 1, RNase T1, S1 Nuclease, RNase H, RNase from Aspergillus, or any combination thereof. An RNase inhibitor provided in a kit may minimally inhibit the polymerase and/or RNase activity of the chimeric RT of the kit. Using an RNase inhibitor provided in a kit which does not inhibit or minimally inhibits the polymerase and/or RNase activity of the chimeric RT can prevent the premature degradation of RNA, e.g., mRNA, for example, before reverse transcription is completed or before a desired quantity of cDNA is obtained.

In some embodiments, the kits disclosed herein comprise an Uracil N-Glycosylase. In PCR assays, for example real-time PCR, UNG can reduce the potential for false positive reactions due to amplicon carryover. Uracil N-Glycosylase (UNG), which cleaves the uracil base from the phosphodiester backbone of uracil-containing DNA, but has no effect on natural (i.e., thymine-containing) DNA, can be used to eliminate carry-over PCR products in real-time PCR. When performing real-time PCR, dUTP can be provided instead of dTTP and the resulting amplicons from amplification can be distinguished from the starting template by the presence of uracil (vs. thymine). Prior to any subsequent amplification, uracil DNA-glycosylase (UNG) can be used to cleave these bases from any contaminating DNA, and therefore only the thymine-containing template remains intact and can be amplified.

In some embodiments, a kit provided herein comprises a DNA polymerase. The DNA polymerase may be provided in the same formulation as the chimeric RT, for example for use in a 1 step RT-PCR reaction. In some embodiments, the DNA polymerase is provided in a formulation different from the chimeric RT, for example for use in a 2 step RT-PCR reaction. In some embodiments, kits disclosed herein may include at least two polymerases. A kit, for example, may contain a main polymerase and a proof-reading polymerase. The main polymerase may be provided at a concentration of at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99%, which may be wt %, vol %, or mol %, or a percentage of the total polymerase in the mix on a molar, mass, or volume basis. The composition may also contain a proof-reading polymerase (e.g., a Tgo based polymerase). The proof-reading polymerase may be present at a concentration of at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99%, which may be wt %, vol %, or mol %, or a percentage of the total polymerase in the mix on a molar, mass, or volume basis. A variety of DNA polymerases are useful in accordance with the present disclosure. Such polymerases include, but are not limited to, Thermus thermophilus (Tth) DNA polymerase, Thermus aquaticus (Taq) DNA polymerase, Thermotoga neapolitana (Tne) DNA polymerase, Thermotoga maritima (Tma) DNA polymerase, Thermococcus litoralis (Tli or VENT™) DNA polymerase, Thermococcus kodakaraensis KOD1 DNA Polymerase, Pyrococcus furiosis (Pfu) DNA polymerase, Pyrococcus species GB-D (DEEPVENT™) DNA polymerase, Pyrococcus woosii (Pwo) DNA polymerase, Bacillus sterothermophilus (Bst) DNA polymerase, Bacillus caldophilus (Bca) DNA polymerase, Sulfolobus acidocaldarius (Sac) DNA polymerase, Thermoplasma acidophilum (Tac) DNA polymerase, Thermus flavus (Tfl/Tub) DNA polymerase, Thermus ruber (Tru) DNA polymerase, Thermus brockianus (DYNAZYME™) DNA polymerase, Methanobacterium thermoautotrophicum (Mth) DNA polymerase, Mycobacterium spp. DNA polymerase (Mtb, Mlep), and mutants, variants and derivatives thereof.

A kit provided herein may comprise a chimeric RT linked to a peptide tag, for example a peptide tag (e.g., SEQ ID NO: 18) that confers thermal stability to the chimeric RT. A DNA polymerase optionally provided in the kit maybe linked to a peptide tag (e.g., SEQ ID NO: 18) that confers thermal stability to the DNA polymerase. An RNase inhibitor optionally provided in a kit may be linked to a peptide tag that confers thermal stability to the RNase inhibitor. A UNG optionally provided in a kit may also be linked to a peptide tag that confers thermal stability to the UNG. Peptide tags that confer thermal stability may allow kits comprising various combinations of the aforementioned components to be stored at room temperature or temperatures higher than room temperature (e.g., temperatures of at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., or at least about 40° C.) for certain periods of time, such as at least 1 day, 2 days, 3 days, 4 days, 5 days, 6 days, 1 week, 2 weeks, 3 weeks, 1 month, 2 months, 3 months, 4 months, 5 months, 6 months or longer than 6 months.

Any of the aforementioned components of a kit can be formulated as a master mix which is ready for use. The master mix can be provided in a single container, e.g., tube. The master mix, in some cases, is provided as a concentrated solution and is ready for use following the appropriate dilutions. For example, the solution may be about 1×, 2×, 3×, 4×, 5×, 10×, or greater than 15× concentration. Solutions formulated at concentrations greater than about 1× can be diluted prior to use. In some embodiments, a master mix for conducting reverse transcription can contain a chimeric reverse transcriptase and optionally a DNA polymerase (e.g., for amplification), optionally an additive (for example, bovine serum albumin or BSA), optionally a DNA tracking dye (for example, Bromophenol blue), optionally a DNA sample loading component (for example, glycerol), optionally a RNase inhibitor, and optionally a UNG. In some embodiments, the components are formulated as at least two master mixes which can be mixed together prior to use. The at least two master mixes can be provided in separate containers, e.g., tubes. Multiple master mixes may, for example, optimize the storage conditions for the various components of the kit.

A kit may further comprise instructions instructing the use of the various components. The instructions may include directions for formulating the reaction sample (including the relevant concentration of chimeric RT, DNA polymerase, RNase inhibitor, UNG, template, primers (reverse and forward), dNTPs, BSA, dyes, and H2O). The instructions may also include recommendations for running the reverse transcription and/or PCR cycle, such as the pre-incubation, denaturation, annealing, and elongation phases. Such instructions may include the temperature conditions and amount of time, temperature ramp rate, and/or number of cycles for each phase. In some embodiments, the foregoing components are added simultaneously at the initiation of the (optional) pre-incubation phase or combined reverse transcription and amplification phase. In some embodiments, components are added in any order prior to or after appropriate time points during the reverse transcription and amplification phases, as required and/or permitted by the reverse transcription and amplification reaction. The enzymes used for nucleic acid amplification can be added to the reaction mixture either prior to the target nucleic acid denaturation step (e.g., a pre-incubation step), following the denaturation step, or following hybridization of the primer to the target RNA or DNA, as determined by their thermal stability and/or other considerations.

### Applications

This disclosure provides methods and compositions for conducting reverse transcription using the RTs provided herein. In some cases, the reverse transcription may include the following steps: (a) in an optional pre-incubation step, RNA template is incubated at an elevated temperature, optionally in the presence of a non-natural RT provided herein; (b) a DNA polymerase domain within a non-natural RT catalyzes the synthesis of cDNA from the RNA template; (c) optionally, RNase within the non-natural RT degrades the RNA in the resulting RNA-DNA hybrids; and (d) DNA-dependent DNA polymerase (either within the non-natural RT or separately added) catalyzes the synthesis of double-stranded DNA.

In some embodiments, reverse transcription is conducted using a chimeric RT provided herein and a DNA polymerase. The DNA polymerase may be provided in the same formulation as the chimeric RT, for example for use in a 1 step RT-PCR reaction. In some embodiments, the DNA polymerase is provided in a formulation different from the chimeric RT, for example for use in a 2 step RT-PCR reaction.

In some embodiments, a composition for conducting 1 step RT-PCR comprises at least two polymerases. The composition, for example, may comprise a chimeric RT and a DNA polymerase. The chimeric RT may be provided at a concentration of at least about 5%, 10%, 15%, 20%, 25%, 30%, 35%, 40%, 45%, 50%, 55%, 60%, 65%, 70%7, 75%, 80%, 85%, 90%, 95%, 96%, 97%, 98%, or 99%, which may be wt %, vol %, or mol %, or a percentage of the total polymerase in the mix on a molar, mass, or volume basis. DNA polymerase may be present at a concentration of at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99%, which may be wt %, vol %, or mol %, or a percentage of the total polymerase in the mix on a molar, mass, or volume basis. A variety of DNA polymerases are useful in accordance with the present disclosure. Such polymerases include, but are not limited to, Thermus thermophilus (Tth) DNA polymerase, Thermus aquaticus (Taq) DNA polymerase, Thermotoga neapolitana (Tne) DNA polymerase, Thermotoga maritima (Tma) DNA polymerase, Thermococcus litoralis (Tli or VENT™) DNA polymerase, Thermococcus kodakaraensis KOD1 DNA Polymerase, Pyrococcus furiosis (Pfu) DNA polymerase, Pyrococcus species GB-D (DEEPVENT™) DNA polymerase, Pyrococcus woosii (Pwo) DNA polymerase, Bacillus sterothermophilus (Bst) DNA polymerase, Bacillus caldophilus (Bca) DNA polymerase, Sulfolobus acidocaldarius (Sac) DNA polymerase, Thermoplasma acidophilum (Tac) DNA polymerase, Thermus flavus (Tfl/Tub) DNA polymerase, Thermus ruber (Tru) DNA polymerase, Thermus brockianus (DYNAZYME™) DNA polymerase, Methanobacterium thermoautotrophicum (Mth) DNA polymerase, Mycobacterium spp. DNA polymerase (Mtb, Mlep), and mutants, variants and derivatives thereof.

cDNA molecules (single-stranded or double-stranded) may be produced from a variety of nucleic acid template molecules using non-natural (e.g., chimeric) RTs described herein. For example, nucleic acid template molecules may include single-stranded or double-stranded RNA (e.g., messenger RNA (mRNA), transfer RNA (tRNA) and ribosomal RNA (rRNA) molecules), as well as double-stranded DNA:RNA hybrids. Prior to reverse transcription and amplification, double-stranded molecules can be treated, for example by heat denaturation, to yield single-stranded molecules.

Nucleic acid template (e.g., RNA template) may be obtained from natural sources, such as a variety of cells, tissues, organs or organisms. Cells that may be used as sources of nucleic acid molecules may be prokaryotic (bacterial cells, including but not limited to those of species of the genera Escherichia, Bacillus, Serratia, Salmonella, Staphylococcus, Streptococcus, Clostridium, Chlamydia, Neisseria, Treponema, Mycoplasma, Borrelia, Legionella, Pseudomonas, Mycobacterium, Helicobacter, Erwinia, Agrobacterium, RMzobium, Xanthomonas and Streptomyces) or eukaryotic (including fungi, plants, protozoans and other parasites, and animals including insects, nematodes, and mammals such as human cells).

In some cases, nucleic acid template is obtained or derived from mammalian somatic cells. Mammalian somatic cells that maybe used as sources of nucleic acids include blood cells (e.g., reticulocytes and leukocytes), endothelial cells, epithelial cells, neuronal cells (e.g., from the central or peripheral nervous systems), muscle cells (e.g., myocytes and myoblasts from skeletal, smooth or cardiac muscle), connective tissue cells (e.g., fibroblasts, adipocytes, chondrocytes, chondroblasts, osteocytes and osteoblasts) and other stromal cells (e.g., macrophages, dendritic cells, Schwann cells). In some cases, nucleic acid template is obtained or derived from mammalian germ cells (e.g., spermatocytes and oocytes). In some cases, nucleic acid template is obtained or derived from the progenitors, precursors and stem cells that give rise to the aforementioned somatic and germ cells. Nucleic acids can also be obtained or derived from mammalian tissues or organs such as the brain, kidney, liver, pancreas, blood, bone marrow, muscle, nervous, skin, genitourinary, circulatory, lymphoid, gastrointestinal and connective tissue sources, as well as those derived from a mammalian (including human) embryo or fetus.

Any of the above prokaryotic or eukaryotic cells, tissues and organs may be normal, diseased, transformed, established, progenitors, precursors, fetal or embryonic. Diseased cells may, for example, include those involved in infectious diseases such as those caused by bacteria, fungi or yeast, viruses (e.g., HIV, HTLV, herpes, hepatitis and the like) or parasites; in genetic or biochemical pathologies (e.g., cystic fibrosis, hemophilia, Alzheimer's disease, muscular dystrophy or multiple sclerosis); or in cancerous tissues.

In some embodiments, the RTs provided herein can be used to detect the presence of organisms in a sample, for example pathogenic organisms. The RTs provided herein may be thermal stable or have high specific activity, and may be particularly useful for clinical applications in hot or tropical climates, for example where temperatures are at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., or at least about 40° C.

The RTs provided herein may be thermally stable or have high specific activity, and may be particularly useful for cases where the RTs are to be transported and/or stored at elevated temperatures (e.g., temperatures of at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., or at least about 40° C.) for certain periods of time (e.g., for at least about a day, at least about 2 days, at least about 3 days, at least about 4 days, at least about 5 days, at least about 6 days, at least about 1 week, at least about 2 weeks, at least about 3 weeks, at least about 1 month, at least about 2 months, at least about 3, at least about 4 months, at least about 5 months, at least about 6 months or longer).

The RTs provided herein may be particularly useful in applications for detecting pathogenic microbes, e.g., disease-causing bacteria. Such bacteria that can cause disease include diphtheria (e.g., Corynebacterium diphtheria), pertussis (e.g., Bordetella pertussis), anthrax (e.g., Bacillus anthracia), typhoid, plague, shigellosis (e.g., Shigella dysenteriae), botulism (e.g., Clostridium botulinum), tetanus (e.g., Clostridium tetani), tuberculosis (e.g., Mycobacterium tuberculosis), bacterial pneumonias (e.g., Haemophilus influenzae), cholera (e.g., Vibrio cholerae), salmonellosis (e.g., Salmonella typhi), peptic ulcers (e.g., Helicobacter pylori), Legionnaire's Disease (e.g. Legionella spp.), and Lyme disease (e.g. Borrelia burgdorferi). Other pathogenic bacteria include Clostridium perfringens, Clostridium difficile, Pseudomonas aeruginosa, Staphylococcus aureus, and Streptococcus pyogenes. Further examples of bacteria include Staphylococcus epidermidis, Staphylococcus sp., Streptococcus pneumoniae, Streptococcus agalactiae, Enterococcus sp., Bacillus cereus, Bifidobacterium bifidum, Lactobacillus sp., Listeria monocytogenes, Nocardia sp., Rhodococcus equi, Erysipelothrix rhusiopathiae, Propionibacterium acnes, Actinomyces sp., Mobiluncus sp., Peptostreptococcus sp., Neisseria gonorrhoeae, Neisseria meningitides, Moraxella catarrhalis, Veillonella sp., Actinobacillus actinomycetemcomitans, Acinetobacter baumannii, Brucella sp., Campylobacter sp., Capnocytophaga sp., Cardiobacterium hominis, Eikenella corrodens, Francisella tularensis, Haemophilus ducreyi, Helicobacter pylori, Kingella kingae, Legionella pneumophila, Pasteurella multocida, Klebsiella granulomatis, Enterobacteriaceae, Citrobacter sp., Enterobacter sp., Escherichia coli, Klebsiella pneumoniae, Proteus sp., Salmonella enteriditis, Salmonella typhi, Shigella sp., Serratia marcescens, Yersinia enterocolitica, Yersinia pestis, Aeromonas sp., Plesiomonas shigelloides, Vibrio cholerae, Vibrio parahaemolyticus, Vibrio vulnificus, Acinetobacter sp., Flavobacterium sp., Burkholderia cepacia, Burkholderia pseudomallei, Xanthomonas maltophilia, Stenotrophomonas maltophila, Bacteroides fragilis, Bacteroides sp., Prevotella sp., Fusobacterium. sp., and Spirillum minus.

The non-natural RTs may also be used to detect disease-causing fungi. Fungi that can cause disease include Acremoniuin spp., Aspergillus spp., Epidermophytoni spp., Exophiala jeanselmei, Exserohilunm spp., Fonsecaea compacta, Fonsecaea pedrosoi, Fusarium oxysporum, Basidiobolus spp., Bipolaris spp., Blastomyces derinatidis, Candida spp., Cladophialophora carrionii, Coccoidiodes immitis, Conidiobolus spp., Cryptococcus spp., Curvularia spp., Fusarium solani, Geotrichum candidum, Histoplasma capsulatum var. capsulatum, Histoplasma capsulatum var. duboisii, Hortaea werneckii, Lacazia loboi, Lasiodiplodia theobromae, Leptosphaeria senegalenisis, Piedra iahortae, Pityriasis versicolor, Pseudallesheria boydii, Pyrenochaeta romeroi, Rhizopus arrhizus, Scopulariopsis brevicaulis, Scytalidium dimidiatum, Sporothrix schenckii, Trichophyton spp., Trichosporon spp., Zygomcete fungi, Madurella grisea, Madurella mycetomatis, Malassezia furfur, Microsporum spp., Neotestudina rosatii, Onychocola canadensis, Paracoccidioides brasiliensis, Phialophora verrucosa, Piedraia hortae, Absidia coryinbifera, Rhizomucor pusillus, and Rhizopus arrhizus.

The non-natural RTs may also be used to detect disease-causing viruses. Such viruses that can cause disease include, but are not limited to, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika virus. In some embodiments, the non-natural RTs provided herein are used in assays to detect Zika virus.

The non-natural RTs may also be used to detect disease-causing protozoa. Illustrative examples of protozoa and other parasites that may cause disease can include, but are not limited to, malaria (e.g. Plasmodium falciparum), hookworm, tapeworms, helminths, whipworms, ringworms, roundworms, pinworms, ascarids, filarids, onchocerciasis (e.g., Onchocerca volvulus), schistosomiasis (e.g. Schistosoma spp.), toxoplasmosis (e.g. Toxoplasma spp.), trypanosomiasis (e.g. Trypanosoma spp.), leishmaniasis (Leishmania spp.), giardiasis (e.g., Giardia lamblia), amoebiasis (e.g., Entamoeba histolytica), filariasis (e.g., Brugia malayi), and trichinosis (e.g., Trichinella spiralis).

Uses of Synthesized cDNA

Following reverse transcription, the cDNA products (e.g., partial and/or full-length product) may be used for further analysis or manipulation. The cDNA products can be optionally isolated and/or purified before proceeding. In some embodiments, cDNA produced using chimeric RTs provided herein may be further amplified, for example in one or more amplification reactions. Reverse transcription and nucleic acid amplification may be one-step (e.g., one-step RT-PCR) or two-step (e.g., two-step RT-PCR) reactions. A one-step RT-PCR type reaction may be accomplished in one tube, thereby lowering the possibility of contamination. Such one-step reactions may comprise (a) mixing a nucleic acid template (e.g., mRNA) with chimeric RT and a DNA polymerase and (b) incubating the mixture under conditions sufficient to permit cDNA synthesis and amplification. Two-step RT-PCR reactions may be accomplished in two separate steps. Such a method may comprise (a) mixing a nucleic acid template (e.g., mRNA) with a chimeric RT, (b) incubating the mixture under conditions sufficient to permit cDNA synthesis or first strand synthesis, (c) mixing the reaction mixture in (b) with one or more DNA polymerases and (d) incubating the mixture of step (c) under conditions sufficient to permit amplification. Optional pre-incubation steps may be used with one-step or two-step RT-PCR. For amplification of long nucleic acid molecules (e.g., greater than about 3-5 kb in length), a combination of DNA polymerases may be used, such as one DNA polymerase having 3′-5′ exonuclease activity and another DNA polymerase being reduced in 3′-5′ exonuclease activity.

Amplification methods which may be used include, but are not limited to, PCR, Strand Displacement Amplification (SDA), and Nucleic Acid Sequence-Based Amplification (NASBA). In some embodiments, amplification by PCR is preferred. In some embodiments, amplification may comprise isothermal amplification methods such as loop-mediated isothermal amplification (LAMP).

When RT-PCR is performed as two steps, the first step of reverse transcription can be conducted in a buffer optimized for RT activity and then a second step of PCR in another buffer condition optimized for PCR activity. Although these two steps can theoretically be combined, finding a single set of reaction conditions suitable for both steps can be challenging. For example, performing reverse transcription and amplification in one step may require an RT to have sufficient sensitivity and speed under conditions optimized for the amplification step; sufficient tolerance to high salt and to other potential inhibitors that may carry over from previous RNA sample preparation processes; and enhanced thermal stability. Performing reverse transcription at elevated temperatures with RTs having enhanced thermal stability may increase the sensitivity and/or speed of reverse transcription by minimizing RNA secondary structure.

As used herein, the terms “% identity” and “% identical” with reference to a sequence, such as a polynucleotide sequence or a polypeptide sequence, refer to comparisons among polynucleotides and polypeptides when the sequences are optimally aligned over a comparison window. The portion of the polynucleotide or polypeptide sequence in the comparison window may comprise additions or deletions (e.g., gaps) as compared to the reference sequence (which does not comprise additions or deletions) for optimal alignment of the two sequences. The percentage can be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Homology can be evaluated using any of the variety of available sequence comparison algorithms and programs. Such algorithms and programs include, but are not limited to, TBLASTN, BLASTP, FASTA, TFASTA, CLUSTALW, FASTDB. In some cases, protein and nucleic acid sequence homologies can be evaluated using the Basic Local Alignment Search Tool (“BLAST”).

As used herein, the term “or” means “and/or” unless stated otherwise.

The term “about” as used herein, unless otherwise indicated, refers to a value that is no more than 10% above or below the value being modified by the term. For example, the term “about −20° C.” means a range of from −22° C. to −18° C., including −22° C. and −18° C. As another example, “about 1 hour” means a range of from 54 minutes to 66 minutes, including 54 minutes and 66 minutes.

As used herein, the term “derived from” in connection with deriving a polypeptide (e.g., polypeptide domain) or a polynucleotide (e.g., polynucleotide domain) from a given source (e.g., a biological organism or microbe) may generally mean that the polypeptide or polynucleotide is identical to or a variant of (e.g., at least 50% identical) a polynucleotide or polypeptide sequence naturally present in the source organism. The term “derived from” is not meant to limit the method by which the molecule is made which can be, for example, by chemical synthesis or recombinant means and may, in many instances, occur without direct reference to the identity of the source organism.

### EXAMPLES

### Example 1—First Strand cDNA Synthesis

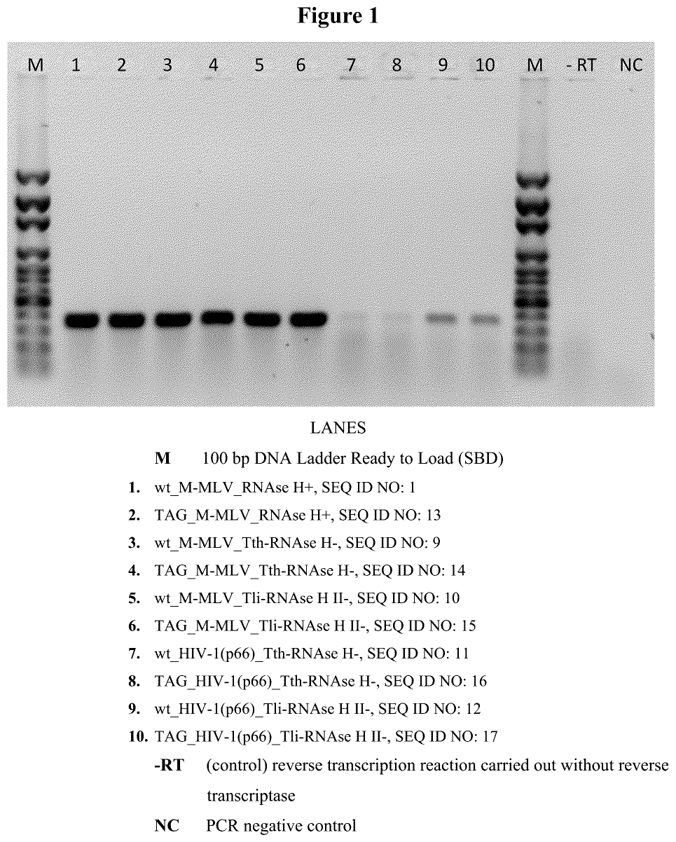

First strand cDNA synthesis was performed on 0.4 μg of total RNA with oligo(dT), at 37° C. for 60 minutes (min) using chimeric reverse transcriptases of the present disclosure. PCR amplification of cDNA was performed using DNA polymerase mix (HOT FIREPol© Blend Master Mix, Solis BioDyne) and 02M primers (˜365 bp PCR product). The DNA product was examined on an agarose gel (FIG. 1).

Chimeric reverse transcriptases having a polymerase domain derived from M-MLV, both with and without a stabilizing peptide tag, possessed enzymatic activity (as indicated by the presence of amplification product on the agarose gel, lanes 3-6) whereas chimeric reverse transcriptases having a polymerase domain derived from HIV-1, both with and without a stabilizing peptide tag, exhibited diminished enzymatic activity (as indicated by minimal amplification product on the agarose gel, lanes 7-10).

### Example 2—Molecular Weight Analysis of Chimeric Reverse Transcriptases

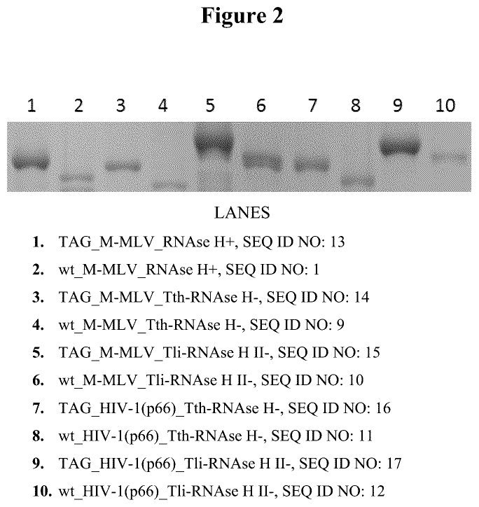

The molecular weight of chimeric reverse transcriptases disclosed herein was analyzed on a PageBlue stained 6% SDS-Polyacrylamide Gel (FIG. 2).

### Example 3—Reverse Transcription Efficiency of Chimeric RTs Following Pre-Incubation

Chimeric reverse transcriptases were pre-incubated for 15 minutes at 55° C., 60° C., 65° C. and 70° C. Pre-incubation can be used, for example, to minimize the presence of secondary structure that may be present in RNA template and interfere with reverse transcription. However, proteins, such as enzymes, that are not thermostable may lose their activity during pre-incubation.

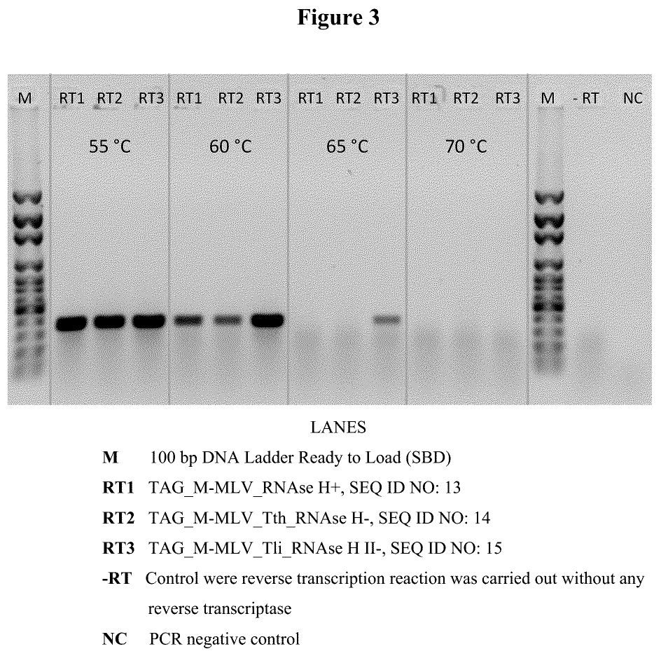

After pre-incubation, first strand cDNA synthesis was performed on 0.4 μg of total RNA with oligo(dT) at 37° C. for 60 min. PCR amplification of cDNA was performed using DNA polymerase mix (HOT FIREPol© Blend Master Mix, Solis BioDyne) and 02M primers. The DNA product was examined on an agarose gel (FIG. 3).

Chimeric reverse transcriptases having a polymerase domain derived from M-MLV possessed enzymatic activity after pre-incubation at 55° C. and 60° C., as indicated by the presence of amplification product on the agarose gel. Chimeric reverse transcriptases having a polymerase domain derived from M-MLV and an RNase H II domain derived from T. litoralis possessed detectable enzymatic activity after pre-incubation at 65° C.

### Example 4—Specific Activity of Chimeric RTs Compared to Wild-Type

In this example, a tagged, chimeric reverse transcriptase having a polymerase domain derived from M-MLV and an RNase H II domain derived from T. litoralis (TAG_M-MLV_Tli_RNase H II-, SEQ ID NO: 15) exhibited higher specific activity compared to tagged, wild-type M-MLV reverse transcriptase.

One unit of reverse transcriptase (RT) can refer to the amount of enzyme that incorporates 1 nanomol (nmol) of dTTP into acid-precipitable material in 10 minutes at 37° C. using poly(rA)-oligo(dT) as template-primer in a total reaction volume of 50 μL. For wild-type M-MLV, 1 μg of enzyme usually corresponds to 200 U/μL of RT activity (˜200 U/μg).

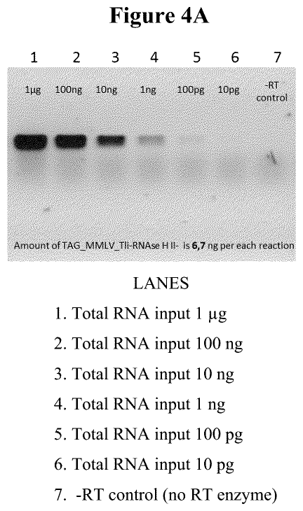

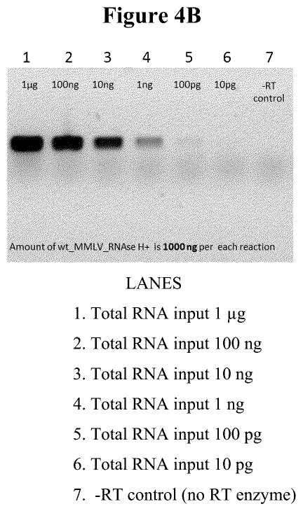

First strand cDNA synthesis was performed on a dilution series (10-fold) of total RNA with oligo(dT) at 37° C. for 60 minutes with wild-type M-MLV or the chimeric reverse transcriptase (SEQ ID NO: 15). PCR amplification of cDNA was performed using DNA polymerase mix (HOT FIREPol® Blend Master Mix, Solis BioDyne) and β2M primers. The PCR product (˜365 bp) was examined on an agarose gel. For the same reaction conditions, first strand cDNA synthesis by ˜6.7 ng of chimeric RT and then PCR amplification of cDNA is able to yield similar amounts of product (FIG. 4A) compared to first strand cDNA synthesis by ˜1000 ng of wild-type M-MLV (˜150× the amount of chimeric RT used) and subsequent PCR amplification (FIG. 4B). The results of FIGS. 4A and 4B demonstrate that decreased amounts of chimeric RT have minimal effect on cDNA quantity and for a broad range of input RNA (e.g., from 10 μg to 1 μg). The specific activity of the chimeric RT of SEQ ID NO: 15 can be estimated to be about 200 U/μg×150≈30,000 U/μg.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-64. (canceled)

65. A non-natural enzyme comprising a first domain and a second domain wherein:
(a) the first domain is a non-ribonuclease enzyme domain; and
(b) the second domain is a ribonuclease domain comprising a sequence having at least 80% sequence identity to SEQ ID NO: 7 or 8, wherein the first domain and the second domain are not derived from a same organism.

66. The non-natural enzyme of claim 65, wherein the ribonuclease domain is an extremophile RNase domain.

67. The non-natural enzyme of claim 65, wherein the ribonuclease domain is a Thermococcus litoralis RNase H domain.

68. The non-natural enzyme of claim 65, wherein the ribonuclease domain is a Thermococcus thermophilus RNase H domain

69. The non-natural enzyme of claim 65, wherein the ribonuclease domain is an RNase H II domain.

70. The non-natural enzyme of claim 69, wherein the RNase H II domain is a mutated RNase H II domain.

71. The non-natural enzyme of claim 70, wherein the mutated RNase H II domain comprises at least one mutation in an active site of the RNase H II domain.

72. The non-natural enzyme of claim 71, wherein the at least one mutation in the active site of the RNAse H II domain is an amino acid substitution, insertion, or deletion.

73. The non-natural enzyme of claim 65, wherein the ribonuclease domain comprises an active site comprising a mutation at least one of residues 7 (D), 8 (E), 105(D), and 135 (D) of relative to SEQ ID NO:8 to a non-wild-type residue.

74. The non-natural enzyme of claim 65, wherein the ribonuclease domain comprises an amino acid sequence having at least 85% sequence identity to SEQ ID NO: 22.

75. The non-natural enzyme of claim 65, wherein the ribonuclease domain comprises an amino acid sequence having at least 95% sequence identity to SEQ ID NO: 22.

76. The non-natural enzyme of claim 65, wherein the ribonuclease domain comprises an amino acid sequence having at least 85% sequence identity to SEQ ID NO: 21.

77. The non-natural enzyme of claim 65, wherein the ribonuclease domain comprises an amino acid sequence having at least 95% sequence identity to SEQ ID NO: 21.

78. The non-natural enzyme of claim 65, wherein the first domain is linked to an N-terminus of the second domain.

79. The non-natural enzyme of claim 65, wherein the first domain is a polymerase domain.

80. The non-natural enzyme of claim 79, wherein the polymerase domain is derived from a virus, an avian virus, a human virus, a murine virus, a retrovirus, a lentivirus, or a gammaretrovirus.

81. The non-natural enzyme of claim 79, wherein the polymerase domain is derived from a viral reverse transcriptase selected from the group consisting of: a Moloney Murine Leukemia Virus (M-MLV) reverse transcriptase, a Rous Sarcoma Virus (RSV) reverse transcriptase, an Avian Myeloblastosis Virus (AMV) reverse transcriptase, a Myeloblastosis Associated Virus (MAV) reverse transcriptase, a Rous Associated Virus (RAV) reverse transcriptase, and a Human Immunodeficiency Virus 1 (HIV-1) reverse transcriptase.

82. The non-natural enzyme of claim 65, wherein the polymerase is derived from a M-MLV reverse transcriptase.

83. The non-natural enzyme of claim 65, wherein the first domain is derived from a polymerase and the polymerase is a DNA polymerase.

84. The non-natural enzyme of claim 65, wherein the first domain is derived from a polymerase and the polymerase is an RNA-dependent DNA polymerase.

85. The non-natural enzyme of claim 65, wherein the first domain is derived from a polymerase and the polymerase is a DNA-dependent DNA polymerase.


SEQUENCE LISTING (27 sequences)

SEQ ID NO: 1 - protein, 671 aa, Murine leukemia virus
LNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLI

SEQ ID NO: 2 - protein, 510 aa, Murine leukemia virus
LNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTD

SEQ ID NO: 3 - protein, 161 aa, Murine leukemia virus
QPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLI

SEQ ID NO: 4 - protein, 562 aa, Human immunodeficiency virus 1
NFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVL

SEQ ID NO: 5 - protein, 437 aa, Human immunodeficiency virus 1
NFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIV

SEQ ID NO: 6 - protein, 125 aa, Human immunodeficiency virus 1
GAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVL

SEQ ID NO: 7 - protein, 166 aa, Thermus thermophilus
MNPSPRKRVALFTDGACLGNPGPGGWAALLRFHAHEKLLSGGEACTTNNRMELKAAIEGLKALKEPCEVDLYTDSHYLKKAFTEGWLEGWRKRGWRTAEGKPVKNRDLWEALLLAMAPHRVRFHFVKGHTGHPENERVDREARRQAQSQAKTPCPPRAPTLFHEEA

SEQ ID NO: 8 - protein, 224 aa, Thermococcus litoralis
MNLGGIDEAGRGPVIGPLVIAAVVVDESRMQELEALGVKDSKKLTPKRREELFEEIVQIVDDHVIIQLSPEEIDGRDGTMNELEIENFAKALNSLKVKPDVLYIDAADVKEKRFGDIIGERLSFSPKIIAEHKADSKYIPVAAASILAKVTRDRAIEKLKELYGEIGSGYPSDPNTRRFLEEYYKAHGEFPPIVRKSWKTLRKIEEKLKAKKTQPTILDFLKKP

SEQ ID NO: 9 - protein, 680 aa, synthetic construct
  REGION 1..680: note = source = /note="Description of Artificial Sequence: Synthetic polypeptide"
LNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDNPSPRKRVALFTNGACLGNPGPGGWAALLRFHAHEKLLSGGEACTTNNRMELKAAIEGLKALKEPCEVDLYTDSHYLKKAFTEGWLEGWRKRGWRTAEGKPVKNRDLWEALLLAMAPHRVRFHFVKGHTGHPENERVDREARRQAQSQAKTPCPPRAPTLFHEEA

SEQ ID NO: 10 - protein, 738 aa, synthetic construct
  REGION 1..738: note = source = /note="Description of Artificial Sequence: Synthetic polypeptide"
LNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDKLGGINQAGRGPVIGPLVIAAVVVDESRMQELEALGVKDSKKLTPKRREELFEEIVQIVDDHVIIQLSPEEIDGRDGTMNELEIENFAKALNSLKVKPDVLYIDAADVKEKRFGDIIGERLSFSPKIIAEHKADSKYIPVAAASILAKVTRDRAIEKLKELYGEIGSGYPSDPNTRRFLEEYYKAHGEFPPIVRKSWKTLRKIEEKLKAKKTQPTILDFLKKP

SEQ ID NO: 11 - protein, 602 aa, synthetic construct
  REGION 1..602: note = source = /note="Description of Artificial Sequence: Synthetic polypeptide"
NFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVNPSPRKRVALFTNGACLGNPGPGGWAALLRFHAHEKLLSGGEACTTNNRMELKAAIEGLKALKEPCEVDLYTDSHYLKKAFTEGWLEGWRKRGWRTAEGKPVKNRDLWEALLLAMAPHRVRFHFVKGHTGHPENERVDREARRQAQSQAKTPCPPRAPTLFHEEA

SEQ ID NO: 12 - protein, 660 aa, synthetic construct
  REGION 1..660: note = source = /note="Description of Artificial Sequence: Synthetic polypeptide"
NFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVKLGGINQAGRGPVIGPLVIAAVVVDESRMQELEALGVKDSKKLTPKRREELFEEIVQIVDDHVIIQLSPEEIDGRDGTMNELEIENFAKALNSLKVKPDVLYIDAADVKEKRFGDIIGERLSFSPKIIAEHKADSKYIPVAAASILAKVTRDRAIEKLKELYGEIGSGYPSDPNTRRFLEEYYKAHGEFPPIVRKSWKTLRKIEEKLKAKKTQPTILDFLKKP

SEQ ID NO: 13 - protein, 707 aa, synthetic construct
  REGION 1..707: note = source = /note="Description of Artificial Sequence: Synthetic polypeptide"
MIDLQRPQAATMDSRHHHHHHPWDYKDDDDKPRWNSLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLI

SEQ ID NO: 14 - protein, 716 aa, synthetic construct
  REGION 1..716: note = source = /note="Description of Artificial Sequence: Synthetic polypeptide"
MIDLQRPQAATMDSRHHHHHHPWDYKDDDDKPRWNSLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDNPSPRKRVALFTNGACLGNPGPGGWAALLRFHAHEKLLSGGEACTTNNRMELKAAIEGLKALKEPCEVDLYTDSHYLKKAFTEGWLEGWRKRGWRTAEGKPVKNRDLWEALLLAMAPHRVRFHFVKGHTGHPENERVDREARRQAQSQAKTPCPPRAPTLFHEEA

SEQ ID NO: 15 - protein, 774 aa, synthetic construct
  REGION 1..774: note = source = /note="Description of Artificial Sequence: Synthetic polypeptide"
MIDLQRPQAATMDSRHHHHHHPWDYKDDDDKPRWNSLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDKLGGINQAGRGPVIGPLVIAAVVVDESRMQELEALGVKDSKKLTPKRREELFEEIVQIVDDHVIIQLSPEEIDGRDGTMNELEIENFAKALNSLKVKPDVLYIDAADVKEKRFGDIIGERLSFSPKIIAEHKADSKYIPVAAASILAKVTRDRAIEKLKELYGEIGSGYPSDPNTRRFLEEYYKAHGEFPPIVRKSWKTLRKIEEKLKAKKTQPTILDFLKKP

SEQ ID NO: 16 - protein, 638 aa, synthetic construct
  REGION 1..638: note = source = /note="Description of Artificial Sequence: Synthetic polypeptide"
MIDLQRPQAATMDSRHHHHHHPWDYKDDDDKPRWNSNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVNPSPRKRVALFTNGACLGNPGPGGWAALLRFHAHEKLLSGGEACTTNNRMELKAAIEGLKALKEPCEVDLYTDSHYLKKAFTEGWLEGWRKRGWRTAEGKPVKNRDLWEALLLAMAPHRVRFHFVKGHTGHPENERVDREARRQAQSQAKTPCPPRAPTLFHEEA

SEQ ID NO: 17 - protein, 696 aa, synthetic construct
  REGION 1..696: note = source = /note="Description of Artificial Sequence: Synthetic polypeptide"
MIDLQRPQAATMDSRHHHHHHPWDYKDDDDKPRWNSNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVKLGGINQAGRGPVIGPLVIAAVVVDESRMQELEALGVKDSKKLTPKRREELFEEIVQIVDDHVIIQLSPEEIDGRDGTMNELEIENFAKALNSLKVKPDVLYIDAADVKEKRFGDIIGERLSFSPKIIAEHKADSKYIPVAAASILAKVTRDRAIEKLKELYGEIGSGYPSDPNTRRFLEEYYKAHGEFPPIVRKSWKTLRKIEEKLKAKKTQPTILDFLKKP

SEQ ID NO: 18 - protein, 36 aa, synthetic construct
  REGION 1..36: note = source = /note="Description of Artificial Sequence: Synthetic polypeptide"
MIDLQRPQAATMDSRHHHHHHPWDYKDDDDKPRWNS

SEQ ID NO: 19 - protein, 5 aa, synthetic construct
  REGION 1..5: note = source = /note="Description of Artificial Sequence: Synthetic peptide"
DDDDK

SEQ ID NO: 20 - protein, 21 aa, synthetic construct
  REGION 1..21: note = source = /note="Description of Artificial Sequence: Synthetic peptide"
HHHHHHPWDYKDDDDKPRWNS

SEQ ID NO: 21 - protein, 166 aa, Thermus thermophilus
MNPSPRKRVALFTNGACLGNPGPGGWAALLRFHAHEKLLSGGEACTTNNRMELKAAIEGLKALKEPCEVDLYTDSHYLKKAFTEGWLEGWRKRGWRTAEGKPVKNRDLWEALLLAMAPHRVRFHFVKGHTGHPENERVDREARRQAQSQAKTPCPPRAPTLFHEEA

SEQ ID NO: 22 - protein, 224 aa, Thermococcus litoralis
MNLGGINQAGRGPVIGPLVIAAVVVDESRMQELEALGVKDSKKLTPKRREELFEEIVQIVDDHVIIQLSPEEIDGRDGTMNELEIENFAKALNSLKVKPDVLYIDAADVKEKRFGDIIGERLSFSPKIIAEHKADSKYIPVAAASILAKVTRDRAIEKLKELYGEIGSGYPSDPNTRRFLEEYYKAHGEFPPIVRKSWKTLRKIEEKLKAKKTQPTILDFLKKP

SEQ ID NO: 23 - protein, 895 aa, Rous sarcoma virus
TVALHLAIPLKWKPDHTPVWIDQWPLPEGKLVALTQLVEKELQLGHIVPSLSCWNTPVFVIRKASGSYRLLHDLRAVNAKLVPFGAVQQGAPVLSALPRGWPLMVLDLKDCFFSIPLAEQDREAFAFTLPSVNNQAPARRFQWKVLPQGMTCSPTICQLVVGQVLEPLRLKHPSLCMLHYMDDLLLAASSHDGLEAAGEEVISTLERAGFTISPDKVQREPGVQYLGYKLGSTYVAPVGLVAEPRIATLWDVQKLVGSLQWLRPALGIPPRLMGPFYEQLRGSDPNEAREWNLDMKMAWREIVQLSTTAALERWDPALPLEGAVARCEQGAIGVLGQGLSTHPRPCLWLFSTQPTKAFTAWLEVLTLLITKLRASAVRTFGKEVDILLLPACFREDLPLPEGILLALKGFAGKIRSSDTPSIFDIARPLHVSLKVRVTDHPVPGPTVFTDASSSTHKGVVVWREGPRWEIKEIADLGASVQQLEARAVAMALLLWPTAPTNVVTDSAFVAKMLLKMGQEGVPSTAAAFILEDALSQRSAMAAVLHVRSHSEVPGFFTEGNDVADSKATFQAYPLREAKDLHTALHIGPRALSKACNISMQQAREVVQTCPHCNSAPALEAGVNPRGLGPLQIWQTDFTLEPRMAPRSWLAVTVDTASSAIVVTQHGRVTSVAAQHHWATAIAVLGRPKAIKTDNGSCFTSKSTREWLARWGIAHTTGIPGNSQGQAMVERANRLLKDRIRVLAEGDGFMKRIPTSKQGELLAKAMYALNHFERGENTKTPIQKHWRPTVLTEGPPVKIRIETGEWEKGWNVLVWGRGYAAVKNRDTDKVIWVPSRKVKPDITQKDEVTKKDEASPLFAGISDWIPWEDEQEGLQGETASNKQERPGEDTLAANES

SEQ ID NO: 24 - protein, 900 aa, unidentified
RATVLTVALHLAIPLKWKPNHTPVWIDQWPLPEGKLVALTQLVEKELQLGHIEPSLSCWNTPVFVIRKASGSYRLLHDLRAVNAKLVPFGAVQQGAPVLSALPRGWPLMVLDLKDCFFSIPLAEQDREAFAFTLPSVNNQAPARRFQWKVLPQGMTCSPTICQLIVGQILEPLRLKHPSLRMLHYMDDLLLAASSHDGLEAAGEEVISTLERAGFTISPDKVQREPGVQYLGYKLGSTYVAPVGLVAEPRIATLWDVQKLVGSLQSVRPALGIPPRLMGPFYEQLRGSDPNEAREWNLDMKMAWREIVQLSTTAALERWDPALPLEGAVARCEQGAIGVLGQGLSTHPRPCLWLFSTQPTKAFTAWLEVLTLLITKLRASAVRTFGKEVDILLLPACFREDLPLPEGILLALRGFAGKIRSSDTPSIFDIARPLHVSLKVRVTDHPVPGPTVFTDASSSTHKGVVVWREGPRWEIKEIADLGASVQQLEARAVAMALLLWPTTPTNVVTDSAFVAKMLLKMGQEGVPSTAAAFILEDALSQRSAMAAVLHVRSHSEVPGFFTEGNDVADSQATFQAYPLREAKDLHTALHIGPRALSKACNISMQQAREVVQTCPHCNSAPALEAGVNPRGLGPLQIWQTDFTLEPRMAPRSWLAVTVDTASSAIVVTQHGRVTSVAAQHHWATAIAVLGRPKAIKTDNGSCFTSKSTREWLARWGIAHTTGIPGNSQGQAMVERANRLLKDKIRVLAEGDGFMKRIPTSKQGELLAKAMYALNHFERGENTKTPIQKHWRPTVLTEGPPVKIRIETGEWEKGWNVLVWGRGYAAVKNRDTDKVIWVPSRKVKPDITQKDEVTKKDEASPLFAGISDWAPWEGEQEGLQEETASNKQERPGEDTPAANES

SEQ ID NO: 25 - protein, 901 aa, unidentified
GRATVFTVALHLAIPLKWKPDHTPVWIDQWPLPEGKLVALTQLVEKELQLGHIEPSLSCWNTPVFVIRKASGSYRLLHDLRAVNAKLVPFGAVQQGAPVLSALPRGWPLMVLDLKDCFFSIPLAEQDREAFAFTLPSVNNQAPARRFQWKVLPQGMTCSPTICQLIVGQILEPLRLKHPSLRMLHYMDDLLLAASSHDGLEAAGEEVISTLERAGFTISPDKVQKEPGVQYLGYKLGSTYVAPVGLVAEPRIATLWDVQKLVGSLQSVRPALGIPPRLMGPFYEQLRGSDPNEAREWNLDMKMAWREIVQLSTTAALERWDPALPLEGAVARCEQGAIGVLGQGLSTHPRPCLWLFSTQPTKAFTAWLEVLTLLITKLRASAVRTFGKEVDILLLPACFREDLPLPEGILLALRGFAGKIRSSDTPSIFDIARPLHVSLKVRVTDHPVPGPTVFTDASSSTHKGVVVWREGPRWEIKEIADLGASVQQLEARAVAMALLLWPTTPTNVVTDSAFVAKMLLKMGQEGVPSTAAAFILEDALSQRSAMAAVLHVRSHSEVPGFFTEGNDVADSQATFQAYPLREAKDLHTALHIGPRALSKACNISMQQAREVVQTCPHCNSAPALEAGVNPRGLGPLQIWQTDFTLEPRMAPRSWLAVTVDTASSAIVVTQHGRVTSVAAQHHWATAIAVLGRPKAIKTDNGSCFTSKSTREWLARWGIAHTTGIPGNSQGQAMVERANRLLKDKIRVLAEGDGFMKRIPTSKQGELLAKAVYALNHFERGENTKTPIQKHWRPTVLTEGPPVKIRIETGEWEKGWNVLVWGRGYAAVKNRDTDKVIWVPSRKVKPDITQKDEVTKRDEASPLFAGISDWAPWEGEQEGLQEETASNKQERPGEDTLAANES

SEQ ID NO: 26 - protein, 895 aa, unidentified
TVALHLAIPLKWKPDHTPVWIDQWPLPEGKLVAVTQLVEKELQLGHIEPSLSCWNTPVFVIRKASGSYRLLHDLRAVNAKLVPFGAVQQGAPVLSALPRGWPLMVLDLKDCFFSIPLAEQDREAFAFTLPSVNNQAPARRFQWKVLPQGMTCSPTICQLVVGQVLEPLRLKHPALRMLHYMDDLLLAASSHDGLEAAGKEVIGTLERAGFTISPDKIQREPGVQYLGYKLGSTYVAPVGLVAEPRIATLWDVQKLVGSLQWLRPALGIPPRLMGPFYEQLRGSDPNEAREWNLDMKMAWREIVQLSTTAALERWDPAQPLEGAVARCEQGAIGVLGQGLSTHPRPCLWLFSTQPTKAFTAWLEVLTLLITKLRASAVRTFGKEVDILLLPACFREDLPLPEGILLALRGFAGKIRSSDTPSIFDIARPLHVSLKVRVTDHPVPGPTVFTDASSSTHKGVVVWREGPRWEIKEIVDLGASVQQLEARAVAMALLLWPTTPTNVVTDSAFVAKMLLKMGQEGVPSTAAAFILEDALSQRSAMAAVLHVRSHSEVPGFFTEGNDVADSQATFQAYPLREAKDLHTALHIGPRALSKACNISMQQAREVVQTCPHCNSAPALEAGVNPRGLGPLQIWQTDFTLEPRMAPRSWLAVTVDTASSAIVVTQHGRVTSVAAQHHWATAIAVLGRPKAIKTDNGSCFTSKSTREWLARWGIAHTTGIPGNSQGQAMVERANRLLKDKIRVLAEGDGFMKRIPASKQGELLAKAMYALNHFERGENTKTPVQKHWRPTVLTEGPPVKIRIETGEWEKGWNVLVWGRGYAAVKNRDTDKVIWVPSRKVKPDITQKDEVTKKDEASPLFAGSSDWIPWGDEQEGLQEEAASNKQEGPGEDTLAANES

SEQ ID NO: 27 - protein, 5 aa, synthetic construct
  REGION 1..5: note = source = /note="Description of Artificial Sequence: Synthetic peptide"
EAAAK